(19) United States
(12) Patent Application Publication
(10) Pub. No.: US 2007/0232572 A1
(43) Pub. Date: Oct. 4, 2007

(54) Chemical derivatives as antitelomerase agents which bind specifically to the G-quadruplex DNA structures and their application as a specific anticancer agent

(75) Inventors: Augustin Hittinger, Igny (FR); Thomas Caulfield, Lebanon, NJ (US); Patrick Mailliet, Fontenay Sous Bois (FR); Herve Bouchard, Thiais (FR); Eliane Mandine, Paris (FR); Jean-Louis Mergny, Villejuif (FR); Lionel Guittat, Paris (FR); Jean-Francois Riou, Reims (FR); Dennis Gomez, Reims (FR); Chafke Belmokhtar, Drancy (FR)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignees: Aventis Pharma S.A., Antony (FR); Centre National De La Recherche Scientifique, Paris Cedex (FR); Museum National D'Histoire Naturelle, Paris (FR); Institut Curie, Paris Cedex (FR); Commissariat A L'Energie Atomique, Paris Cedex (FR); Universite De Reims Champagne-Ardenne, Reims Cedex (FR)

(21) Appl. No.: 10/773,806

(22) Filed: Feb. 6, 2004

Related U.S. Application Data
(60) Provisional application No. 60/467,213, filed on May 1, 2003.

(30) Foreign Application Priority Data
Feb. 7, 2003 (FR) 03 01478

(51) Int. Cl. A61K 31/47; A61K 31/497; A61K 31/56; A61K 31/66; C07D 215/00

(52) U.S. Cl. 514/114; 514/182; 514/252.11; 514/255.05; 514/283; 514/313; 544/405; 546/159

(57) ABSTRACT

The present invention relates to cancer therapy and to novel anticancer agents having a mechanism of action which is quite specific. It also relates to novel chemical compounds and their therapeutic application in humans.

This application claims the benefit of U.S. Provisional Application No. 60/476,213 filed May 1, 2003 and right of priority from French Patent Application No. 03 01478, filed Feb. 7, 2003.

The present invention relates to cancer therapy and to novel anticancer agents having a specific mechanism of action. It also relates to a selection of chemical compounds and their therapeutic application in humans.

The present invention relates to the use of novel non-nucleotide chemical compounds which interact with specific structures of deoxyribonucleic acid (DNA) or ribonucleic acid (RNA). These novel compounds consist of a distribution agent linked to two nitrogen-containing heteroaromatic groups of which at least one of the nitrogen atoms is in quaternized form. They are particularly useful for very selectively stabilizing DNA folded into a G-quadruplex structure (guanine tetrads), when the G-quadruplex is formed of either one, two or four DNA strands. These novel compounds are useful in the treatment of cancer and act in particular as telomerase-inhibiting agents.

The therapeutic application of the inhibition of telomerase via the stabilization of these G-quadruplexes may be either the halting of cellular mytosis and the death of rapidly-dividing cells within a period of one to a few weeks by a mechanism of deprotection of telomeric DNA, or the induction of the senescence of cancer cells, following gradual shortening of telomeric DNA (Oncogene 2002, 21, 553-63; Oncogene 2002, 21, 592-97). Another therapeutic application, in the treatment of cancer, of the stabilization of G-quadruplex DNA structures may be carried out by the inactivation of the promoter regions, rich in guanine repeats, of oncogenes such as c-myc (J. Biol. Chem. 2001, 276, 4640-46; Proc. Natl. Acad. Sci. USA 2002, 99, 11593-98), H-ras or h-TERT.

Another therapeutic application, in the treatment of cancer, of the stabilization of G-quadruplex DNA structures may be the inhibition of helicases which are specific for the G-quadruplex DNA structures, and which are involved in mytosis. These helicases are also directly involved in various genetic diseases such as Bloom's syndrome (Cell 1995, 83, 655-66), Werner's syndrome (Science 1996, 272, 258-62), Rothmund-Thomson syndrome (Nature Genetics 1999, 22, 82-4) or ataxia telangiectasia syndrome.

The compounds of the present invention have in particular the advantage, from the therapeutic point of view, of blocking telomerase. From the biological point of view, telomerase allows the addition of repetitive DNA sequences of the T T A G G G type, termed telomeric sequences, at the end of the telomere, during cell division. Through this action telomerase renders the cells immortal. Indeed, in the absence of this enzymatic activity, the cells loses at each division 100 to 150 bases, which rapidly renders it senescent. During the appearance of rapidly-dividing cancer cells, it appeared that these cells had telomers maintained at a stable length during cell division. In these cancer cells, it appeared that telomerase was strongly activated and that it allowed the addition of repetitive units of telomeric sequences at the end of the telomere and therefore allowed preservation of the length of the telomere. It appeared for some time that more than 85% of cancer cells showed positive tests for the presence of telomerase whereas somatic cells do not show this characteristic.

Telomerase is thus a highly coveted target for treating cancer cells. The first obvious approach for blocking telomerase was the use of nucleotide structures (Proc. Natl. Acad. Sci. USA 1996, 93, 2635-39). Various approaches have since been developed for inhibiting telomerase (Curr. Pharm. Des. 2002, 8, 2491-504). Among these approaches, the development of ligands for G-quadruplex DNA is increasingly of interest (Mini Rev. Med. Chem. 2003, 3, 11-21).

It is possible to note the identification of telomestatin, a molecule capable of binding to a G-quadruplex DNA structure in a very specific manner compared with double-stranded DNA (J. Amer. Chem. Soc. 2001, 123, 1262-63). A molecule which is highly selective for the G-quadruplex DNA will have the double advantage of targeting the G-quadruplex DNA structures while avoiding undesirable mechanisms of toxicity which are linked to a nonselective binding to the genome.

Patent WO 0296903 describes the preparation of heterocyclic diamides of the following general formula (I), as ligands for G-quadruplex DNA and their use as telomerase inhibitors:
nitrogen-containing aromatic ring-(NR₃)p-(CO)n-distribution agent-(CO)m-(NR′₃)q-aromatic or nonaromatic ring (I)
with n, m, p and q, which are identical or different, representing the integer 0 or 1,
in which
   the nitrogen-containing aromatic ring represents:
      a quinoline optionally substituted with at least
         one group N(Ra)(Rb) in which Ra and Rb, which are identical or different, represent hydrogen or a C1-C4 alkyl radical or
         one short-chain C1-C4 alkyl or alkoxy group or
      a quinoline possessing a nitrogen atom in quaternary form or
      a benzamidine or
      a pyridine
   the aromatic or nonaromatic ring represents
      a quinoline optionally substituted with at least
         one group N(Ra)(Rb) in which Ra and Rb, which are identical or different, represent hydrogen or a C1-C4 alkyl radical or
         one short-chain C1-C4 alkyl or alkoxy group or
      a quinoline possessing a nitrogen atom in quaternary form or
      a benzamidine or
      a pyridine or
      a phenyl nucleus optionally substituted with a halogen atom, a C1-C4 alkoxy group, a cyano group, a carbonylamino group optionally substituted with one or more C1-C4 alkyl groups, a guanyl group, a C1-C4 alkylthio group, an amino group, a C1-C4 alkylamino group, a C1-C4 dialkylamino group for each alkyl group, a nitro group, a C1-C4 alkyleneamino group or a C2-C4 alkenyleneamino group or
      a mono- or bi- or tricyclic aromatic or nonaromatic heterocyclic nucleus containing 0 to 2 heteroatoms per ring provided that at least one heteroatom has at least one ring optionally substituted with one or more C1-C4 alkyl groups or with C1-C4 alkylene or C2-C4 alkenylene groups
   R₃ and R′₃, which are identical or different, represent independently of each other hydrogen or a C1-C4 alkyl radical
   the distribution agent represents:
      a triazine group optionally substituted with one or more radicals chosen from halogen atoms, alkyl radicals having 1 to 4 carbon atoms and thio, oxy or amino radicals which are themselves optionally substituted with one or more short-chain alkyl chains containing 1 to 4 carbon atoms or
      a 5- or 6-membered heterocyclic radical containing a sulfur, oxygen or nitrogen atom
      a phenyl, —NH-phenyl-NH—, —NH-phenyl-CH2-NH—, —NH—CH2-phenyl-CH2-NH—, —NH—CH2-phenyl-NH—, —CH2-phenyl-CH2-, —CH2-phenyl, -phenyl-CH2-, —CH2-thienyl-, -thienyl-CH2-, or —CH═CH— radical, or
      a diazine group,
   the heterocyclic, phenyl, —NH-phenyl-NH—, —NH-phenyl-CH2-NH—, —NH—CH2-phenyl-CH2-NH—, —NH—CH2-phenyl-NH—, —CH2-phenyl-CH2—, —CH2-phenyl, -phenyl-CH2-, —CH2-thienyl-, -thienyl-CH2-, or —CH═CH— radical, and diazine radicals being optionally substituted with the same groups as the triazine
      it being understood that when the distribution agent represents phenyl optionally substituted with NH₂, that n, m, p and q represent 1 and R₃ and R′₃ represent hydrogen, then the nitrogen-containing aromatic ring and the aromatic ring do not both represent a quinoline which is unsubstituted or substituted on its nitrogen atom with an alkyl radical containing 1 to 6 carbon atoms, or one of its salts and when the distribution agent represents a triazine and p and q both represent the integer 1, then n and m do not both represent the integer 0.

The present invention describes the preparation of heterocyclic diamides of the following general formula (IB) as ligands which are highly specific for G-quadruplex DNA and their use as telomerase inhibitors:
nitrogen-containing aromatic ring possessing a nitrogen atom in quaternary form-(NR₃)p-CO-distribution agent-(CO)m-(NR′₃)q-X-aromatic or nonaromatic ring (IB)
with m, p and q, which are identical or different, representing the integer 0 or 1,
in which
   the nitrogen-containing aromatic ring possessing a quaternary atom represents:
      a quinoline optionally substituted with at least
         one group N(Ra)(Rb) in which Ra and Rb, which are identical or different, represent hydrogen or a C1-C4 alkyl radical or
         one short-chain C1-C4 alkyl or alkoxy group or
      and in which the nitrogen atom is quaternized with a C1-C4 alkyl chain optionally substituted with a hydroxyl, carboxyl, C1-C4 alkoxy, C1-C4 alkylthio, amino, C1-C4 alkylamino or C1-C4 dialkylamino radical for each alkyl group,
   the aromatic or nonaromatic ring represents
      a quinoline optionally substituted with at least
         one group N(Ra)(Rb) in which Ra and Rb, which are identical or different, represent hydrogen or a C1-C4 alkyl radical or
         one short-chain C1-C4 alkyl or alkoxy group or
      a quinoline possessing a nitrogen atom in quaternary form or
      a benzamidine or
      a pyridine or
      a phenyl nucleus optionally substituted with a halogen atom, a C1-C4 alkoxy group, a cyano group, a carbonylamino group optionally substituted with one or more C1-C4 alkyl groups, a guanyl group, a C1-C4 alkylthio group, an amino group, a C1-C4 alkylamino group, a C1-C4 dialkylamino group for each alkyl group, a nitro group, a C1-C4 alkyleneamino group or a C2-C4 alkenyleneamino group or
      a mono- or bi- or tricyclic aromatic or nonaromatic heterocyclic nucleus containing 0 to 2 heteroatoms per ring provided that at least one heteroatom has at least one ring optionally substituted with one or more C1-C4 alkyl groups or with C1-C4 alkylene or C2-C4 alkenylene groups, and in which the heteroatom, when it represents a nitrogen atom, may be optionally in quaternary form
   R₃ and R′₃, which are identical or different, represent independently of each other hydrogen or a C1-C4 alkyl or an aralkyl radical in which the alkyl part is C1-C4
   X represents a single bond, or a straight or branched C1-C4 alkyl radical, a C2-C4 alkenyl radical, a C2-C4 alkynyl radical or a phenyl radical
   the distribution agent represents:
      a 5- or 6-membered heterocyclic radical containing a sulfur, oxygen or nitrogen atom
      a phenyl radical or
      a diazine or triazine group,
         the heterocyclic, phenyl, diazine or triazine radicals being optionally substituted with one or more radicals chosen from halogen atoms, alkyl radicals having 1 to 4 carbon atoms and thio, oxy or amino radicals which are themselves optionally substituted with one or more short-chain alkyl chains containing 1 to 4 carbon atoms.

it being understood that for the products of formula (IB) in which X represents a single bond, when the distribution agent represents phenyl optionally substituted with NH₂, m, p and q represent 1 and R₃ and R′₃ represent hydrogen, then the nitrogen-containing aromatic ring and the aromatic ring do not both represent a quinoline which is unsubstituted or substituted on its nitrogen atom with an alkyl radical containing 1 to 6 carbon atoms,

or one of its salts and when the distribution agent represents a triazine and p and q both represent the integer 1 then m does not represent the integer O, Said products of formula (IB) may be in all the possible isomeric forms, such as the racemic, enantiomeric and diastereoisomeric forms, and the addition salts with inorganic or organic acids or with inorganic or organic bases of said products of formula (IB).

In the compounds described in the present invention:
the term alkyl or alk radical denotes a linear or branched radical containing at most 12 carbon atoms chosen from methyl, ethyl, propyl, isopropyl, butyl, isobutyl, sec-butyl, tert-butyl, pentyl, isopentyl, sec-pentyl, tert-pentyl, neopentyl, hexyl, isohexyl, sec-hexyl, tert-hexyl and heptyl, octyl, nonyl, decyl, undecyl and dodecyl radicals, and their linear or branched position isomers.

There may be mentioned more particularly the alkyl radicals having at most 6 carbon atoms and in particular the methyl, ethyl, propyl, isopropyl, n-butyl, isobutyl, tert-butyl, linear or branched pentyl, and linear or branched hexyl radicals.

the term alkenyl radical denotes a linear or branched radical containing at most 12 carbon atoms and preferably 4 carbon atoms chosen, for example, from the following values: ethenyl or vinyl, propenyl or allyl, 1-propenyl, n-butenyl, isobutenyl, 3-methylbut-2-enyl, n-pentenyl, hexenyl, heptenyl, octenyl, cyclohexylbutenyl and decenyl and their linear or branched position isomers.

Among alkenyl values, there may be mentioned more particularly the allyl or butenyl values.
the term alkynyl radical denotes a linear or branched radical containing at most 12 carbon atoms and preferably 4 carbon atoms chosen, for example, from the following values: ethynyl, propynyl or propargyl, butynyl, n-butynyl, isobutynyl, 3-methylbut-2-ynyl, pentynyl or hexynyl and their linear or branched position isomers.

Among the alkynyl values, the propargyl value may be more particularly mentioned.
the term aryl radical denotes radicals which are unsaturated, monocyclic or consist of fused rings, and carbocyclic. As examples of such aryl radical, there may be mentioned phenyl or naphthyl radicals.

There may be mentioned more particularly the phenyl radical.
the term arylalkyl is understood to mean the radicals resulting from the combination of the alkyl radicals mentioned above and optionally substituted and the aryl radicals, also mentioned above and optionally substituted: there may be mentioned for example benzyl, phenylethyl, 2-phenethyl, triphenylmethyl or naphthlenemethyl radicals.
the term heterocyclic radical denotes a carbocyclic radical which is saturated (heterocycloalkyl) or unsaturated (heteroaryl) and consists at most of 6 members interrupted by one or more heteroatoms, which are identical or different, chosen from oxygen, nitrogen or sulfur atoms.

As heterocycloalkyl radicals, there may be mentioned in particular dioxolane, dioxane, dithiolane, thiooxolane, thiooxane, oxiranyl, oxolanyl, dioxolanyl, piperazinyl, piperidinyl, pyrrolidinyl, imidazolidinyl, pyrazolidinyl, morpholinyl or tetrahydrofuryl, tetrahydrothienyl, chromanyl, dihydrobenzofuranyl, indolinyl, piperidinyl, perhydropyranyl, pyrindolinyl, tetrahydroquinolinyl, tetrahydroisoquinolinyl or thioazolidinyl radicals, all these radicals being optionally substituted.

Among the heterocycloalkyl radicals, there may be mentioned in particular the optionally substituted piperazinyl radical, the optionally substituted piperidinyl radical, the optionally substituted pyrrolidinyl radical, the imidazolidinyl radical, the pyrazolidinyl radical, the morpholinyl radical or thioazolidinyl radical. The term heterocycloalkylalkyl radical is understood to mean the radicals in which the heterocycloalkyl and alkyl residues have the above meanings. Among the 5-membered heteroaryl radicals, there may be mentioned the furyl radical such as the 2-furyl radical, the thienyl radical such as the 2-thienyl and 3-thienyl radicals, the pyrrolyl radical, the diazolyl radical, the thiazolyl radical, the thiadiazolyl radical, the thiatriazolyl radical, the isothiazolyl radical, the oxazolyl radical, the oxadiazolyl radical, the 3- or 4-isoxazolyl radical, the imidazolyl radical, the pyrazolyl radical, the isoxazolyl radical.

Among the 6-membered heteroaryl radicals, there may be mentioned in particular the pyridyl radical such as the 2-pyridyl, 3-pyridyl and 4-pyridyl radicals, the pyrimidyl radical, the pyrimidinyl radical, the pyridazinyl radical, the pyrazinyl radical and the tetrazolyl radical.

As fused heteroaryl radicals containing at least one heteroatom chosen from sulfur, nitrogen and oxygen, there may be mentioned, for example, benzothienyl such as 3-benzothienyl, benzofuryl, benzofuranyl, benzopyrrolyl, benzimidazolyl, benzoxazolyl, thionaphtyl, indolyl, purinyl, quinolinyl, isoquinolinyl and naphthyridinyl.

Among the fused heteroaryl radicals, there may be mentioned more particularly the benzothienyl, benzofuranyl, indolyl or quinolinyl, benzimidazolyl, benzothiazolyl, furyl, imidazolyl, indolizinyl, isoxazolyl, isoquinolinyl, isothiazolyl, oxadiazolyl, pyrazinyl, pyridazinyl, pyrazolyl, pyridyl, pyrimidinyl, pyrrolyl, quinazolinyl, 1,3,4-thiadiazolyl, thiazolyl, thienyl and triazolyl radicals, these radicals being optionally substituted as indicated for the heteroaryl radicals.

In the products of formula (IB) as defined above, there may be mentioned more particularly those for which R₃ and R′₃, which are identical or different, represent independently of each other hydrogen or a C1-C4 alkyl or an aralkyl radical in which the alkyl radical is C1-C4, X represents a single bond, a C1-C4 alkyl radical, a C2-C4 alkenyl or alkynyl radical or a phenyl radical, the other substituents being chosen from the values indicated above.

The expression nitrogen-containing aromatic ring is understood to mean in the context of the above formula a heterocycle containing at least one nitrogen atom or an aromatic group containing no heteroatom in the ring but containing at least one nitrogen atom in a hydrocarbon chain linked to the ring, such as for example a guanidino or guanyl chain.

It is evident that the quinoline units may be substituted with any other group which is not involved in the application aimed at; thus acridine or isoquinoline or quinazoline or quinoxaline or phthalazine or benzothiazine or benzoxazine or phenoxazine or phenothiazine groups are included in the definition of the quinoline groups.

Among all the above compounds, those preferred contain a distribution agent chosen from heterocyclic groups such as, for example, pyridyl or thienyl, a phenyl radical a diazine or a triazine. Among the diazine groups, the use of pyridazines is preferred.

Among all the above compounds, those particularly preferred contain a distribution agent which is meta-disubstituted with the groups “nitrogen-containing aromatic ring possessing a nitrogen atom in quaternary form-(NR₃)p-CO” and “(CO)m-(NR′₃)q-aromatic or nonaromatic ring” as defined above, and in which the distribution agent is, in more, optionally substituted by halogen atom.

Among all the above compounds, those preferred contain a distribution agent which is meta-disubstituted with the groups “nitrogen-containing aromatic ring possessing a nitrogen atom in quaternary form-(NR₃)p-CO” and “(CO)m-(NR′₃)q-aromatic or nonaromatic ring” as defined above.

Among the compounds of the present invention, the compounds whose heterocycle in quaternary form is a quinoline are preferred.

Among the compounds of the present invention, the compounds defined above, characterized in that m, p and q represent the integer 1, are in particular preferred.

Among the compounds of the present invention, those particularly most especially preferred are the compounds in which the distribution agent represents a pyridine which is 2,6-disubstituted or a pyridazine which is 2-6-disubstituted with the groups “nitrogen-containing aromatic ring possessing a quaternary nitrogen atom in quaternary form-(NR₃)p-CO” and “(CO)m-(NR′₃)q-aromatic or nonaromatic ring” and in which the quaternized heterocycle is an N-methylquinolinium and in which the distribution agent is, in more, optionally substituted by halogen atom.

Among the compounds of the present invention, those most especially preferred are the compounds in which the distribution agent represents a pyridine which, is 2,6-disubstituted or a pyridazine which is 2-6-disubstituted with the groups “nitrogen-containing aromatic ring possessing a quaternary nitrogen atom in quaternary form-(NR₃)p-CO” and “(CO)m-(NR′₃)q-aromatic or nonaromatic ring” and in which the quaternized heterocycle is an N-methylquinolinium.

The subject of the present invention is in particular especially the products of formula (IB) as defined above, characterized in that they correspond to the formula (Ia) below: 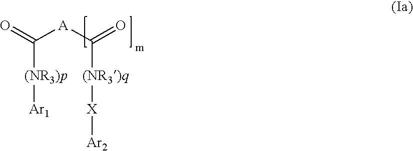
with m, p and q, which are identical or different, representing the integer 0 or 1
A represents:
   a 5- or 6-membered heterocyclic radical containing a sulfur, oxygen or nitrogen atom
   a phenyl radical or
   a diazine or triazine group,
      the heterocyclic, phenyl, diazine or triazine radicals being optionally substituted with one or more radicals chosen from halogen atoms, alkyl radicals having 1 to 4 carbon atoms and thio, oxy or amino radicals which are themselves optionally substituted with one or more short-chain alkyl chains containing 1 to 4 carbon atoms,
      Ar₁ and Ar₂, which are identical or different represent when Ar₁ and Ar₂ are identical, they represent a nitrogen-containing aromatic ring possessing a quaternary atom represented by a quinoline optionally substituted with at least
one group N(Ra)(Rb) in which Ra and Rb, which are identical or different, represent hydrogen or C1-C4 alkyl radical or
one short-chain C1-C4 alkyl or alkoxy group or
and in which the nitrogen atom is quaternized with a C1-C4 alkyl chain optionally substituted with a hydroxyl, carboxyl, C1-C4 alkoxy, C1-C4 alkylthio, amino, C1-C4 alkylamino or C1-C4 dialkylamino radical for each alkyl group,
   when Ar₁ and Ar₂ are different
   Ar₁ represents one of the above possibilities and Ar₂ represents
a phenyl nucleus optionally substituted with a halogen atom, a C1-C4 alkoxy group, a cyano group, a carbonylamino group optionally substituted with one or more C1-C4 alkyl groups, a guanyl group, a C1-C4 alkylthio group, an amino group, a C1-C4 alkylamino group, a C1-C4 dialkylamino group for each alkyl group, a nitro group, a C1-C4 alkyleneamino group or a C2-C4 alkenyleneamino group or
a benzamidine
a pyridyl nucleus
a mono- or bi- or tricyclic aromatic or nonaromatic heterocyclic nucleus containing 0 to 2 heteroatoms per ring provided that at least one heteroatom has at least one ring optionally substituted with one or more C1-C4 alkyl groups or with C1-C4 alkylene or C2-C4 alkenylene groups,
   R₃ and R′₃, which are identical or different, represent independently of each other hydrogen or a C1-C4 alkyl radical or an aralkyl radical in which the alkyl radical is C1-C4
   X represents a single bond, or a C1-C4 alkyl radical, a C2-C4 alkenyl or alkynyl radical or a phenyl radical,
      said products of formula (Ia) may be in all the possible isomeric forms, such as the racemic, enantiomeric and diastereoisomeric forms, and the addition salts with inorganic or organic acids or with inorganic or organic bases of said products of formula (Ia).

The subject of the present invention is thus the products of formula (Ia) as defined above in which X represents a C1-C4 alkyl radical, the other substituents of the products of formula (Ia) being chosen from the values indicated above,

said products of formula (Ia) may be in all the possible isomeric forms, such as the racemic, enantiomeric and diastereoisomeric forms, and the addition salts with inorganic or organic acids or with inorganic or organic bases of said products of formula (Ia).

The subject of the present invention is thus the products of formula (Ia) as defined above, characterized in that A is chosen from heterocyclic groups such as, for example, pyridyl or thienyl, a phenyl radical, a diazine or a triazine as defined above.

The subject of the present invention is thus the products of formula (Ia) as defined above, characterized in that the diazine groups which A may represent are pyrazines.

The subject of the present invention is particularly the products of formula (Ia) as defined above, characterized in that A is meta-disubstituted with the groups “nitrogen-containing aromatic ring possessing a nitrogen atom in quaternary form-(NR₃)p-CO” and “(CO)m-(NR′₃)q-aromatic or nonaromatic ring” as defined above and in which A is, in more, optionally substituted by halogen atom.

The subject of the present invention is the products of formula (Ia) as defined above, characterized in that A is meta-disubstituted with the groups “nitrogen-containing aromatic ring possessing a nitrogen atom in quaternary form-(NR₃)p-CO” and “(CO)m-(NR′₃)q-aromatic or nonaromatic ring” as defined above.

The subject of the present invention is thus the products of formula (Ia) as defined above, characterized in that the heterocycle in quaternary form is a quinoline.

The subject of the present invention is thus particularly the products of formula (Ia) as defined above, characterized in that A represents a pyridine which is 2,6-disubstituted or a pyridazine which is 2-6-disubstituted with the groups “nitrogen-containing aromatic ring possessing a quaternary nitrogen atom in quaternary form-(NR₃)p-CO” and “(CO)m-(NR′₃)q-aromatic or nonaromatic ring” and in which the quaternized heterocycle is an N-methylquinolinium, and in which A is, in more, optionally substituted by halogen atom.

The subject of the present invention is thus the products of formula (Ia) as defined above, characterized in that A represents a pyridine which is 2,6-disubstituted or a pyridazine which is 2-6-disubstituted with the groups “nitrogen-containing aromatic ring possessing a quaternary nitrogen atom in quaternary form-(NR₃)p-CO” and “(CO)m-(NR′₃)q-aromatic or nonaromatic ring” and in which the quaternized heterocycle is an N-methylquinolinium.

The subject of the present invention is thus the products of formula (Ia) as defined above, characterized in that p and q represent the integer 1.

The subject of the present invention is thus the products of formula (Ia) as defined above, characterized in that m, p and q represent the integer 1.

The subject of the present invention is in particular the products of formula (Ia) as defined above, characterized in that Ar₂ represents a group chosen from the following groups: 4-amino- or 4-methylamino-, 4-dimethylamino- or 4-alkoxy-quinolyl or -quinolinium in which the quinolinium nucleus is optionally substituted with one or two methyl groups.

The subject of the present invention is in particular the products of formula (Ia) as defined above, characterized in that R₃ and R′₃ represent hydrogen.

The subject of the present invention is in particular the products of formula (IB) as defined above, including the following names:
bis[(1-methylquinolinio-6-yl)amido]-2,6-pyridinedicarboxylic acid diiodide
2-[(1-methylquinolinio-6-yl)amido]-6-[(4-dimethylamino-1-methylquinaldinio-6-yl)amido]-2,6-pyridinedicarboxylic acid diiodide
bis[(1-methylquinolinio-6-yl)amido]-2,6-pyrazinedicarboxylic acid diiodide
bis[(1-methylquinolinio-6-yl)amido]-1,3-benzenedicarboxylic acid diiodide
bis[(1-methylquinaldinio-6-yl)amido]-2,6-pyridinedicarboxylic acid diiodide
2-[(1-methylquinolinio-6-yl)amido]-6-[(4-aminoquinaldinio-6-yl)amido]-2,6-pyridinedicarboxylic acid iodide, isolated in its imino tautomeric form below:
bis[(1-methylquinaldinio-6-yl)amido]-2,6-benzenedicarboxylic acid diiodide
bis[(1-methylquinolin-6-yl)amido]-2,4-pyridinedicarboxylic acid diiodide
2-[(1-methylquinolinio-6-yl)amido]-6-[(1-methylquinolinio-3-yl)amido]-2,6-pyridinedicarboxylic acid iodide
2-[(1-methylquinolinio-6-yl)amido]-6-[(1-methylquinolinio-5-yl)amido]-2,6-pyridinedicarboxylic acid iodide
bis[(1-methylquinolinio-3-yl)amido]-2,6-pyridinedicarboxylic acid diiodide
2-[(1-methylquinolinio-6-yl)amido]-6-[2(-1-methylpiperidinio-1-yl)ethylamido]-2,6-pyridinedicarboxylic acid diiodide
2,6-pyridinedicarboxylic acid 2-[(1-methylquinolinio-3-yl)amide]-6-[quinolin-3-yl)amide]iodide
2,6-pyridinedicarboxylic acid 2-[(1-methylquinolinio-3-yl)amide]-6-[1-(2-hydroxyethyl)quinolinio-3-yl)amide]iodide
4-bromo-2,6-pyridinedicarboxylic acid bis[(1-methylquinolinio-3-yl)amide]diiodide,
   said products of formula (Ia) may be in all the possible isomeric forms, such as the racemic, enantiomeric and diastereoisomeric forms, and the addition salts with inorganic or organic acids or with inorganic or organic bases of said products of formula (Ia).

The subject of the present invention is thus particularly the products of formula (IB) as defined above including the following names:
bis[(1-methylquinolinio-6-yl)amido]-2,6-pyridinedicarboxylic acid diiodide
2-[(1-methylquinolinio-6-yl)amido]-6-[(4-dimethylamino-1-methylquinaldinio-6-yl)amido]-2,6-pyridinedicarboxylic acid diiodide
bis[(1-methylquinolinio-6-yl)amido]-2,6-pyrazinedicarboxylic acid diiodide
bis[(1-methylquinolinio-6-yl)amido]-1,3-benzenedicarboxylic acid diiodide
bis[(1-methylquinaldinio-6-yl)amido]-2,6-pyridinedicarboxylic acid diiodide
2-[(1-methylquinolinio-6-yl)amido]-6-[(4-aminoquinaldinio-6-yl)amido]-2,6-pyridinedicarboxylic acid iodide, isolated in its imino tautomeric form below:
bis[(1-methylquinaldinio-6-yl)amido]-2,6-benzenedicarboxylic acid diiodide
bis[(1-methylquinolin-6-yl)amido]-2,4-pyridinedicarboxylic acid diiodide
2-[(1-methylquinolinio-6-yl)amido]-6-[(1-methylquinolinio-3-yl)amido]-2,6-pyridinedicarboxylic acid iodide
2-[(1-methylquinolinio-6-yl)amido]-6-[(1-methylquinolinio-5-yl)amido]-2,6-pyridinedicarboxylic acid iodide
bis[(1-methylquinolinio-3-yl)amido]-2,6-pyridinedicarboxylic acid diiodide
2,6-pyridinedicarboxylic acid 2-[(1-methylquinolinio-3-yl)amide]-6-[quinolin-3-yl)amide]iodide
2,6-pyridinedicarboxylic acid 2-[(1-methylquinolinio-3-yl)amide]-6-[1-(2-hydroxyethyl)quinolinio-3-yl)amide]iodide
4-bromo-2,6-pyridinedicarboxylic acid bis[(1-methylquinolinio-3-yl)amide]diiodide
   or the salts or other salts of these compounds said products of formula (IB) may be in all the possible isomeric forms, such as the racemic, enantiomeric and diastereoisomeric forms, and the addition salts with inorganic or organic acids or with inorganic or organic bases of said products of formula (IB).

The subject of the present invention is thus the following product of formula (IB):
2-[(1-methylquinolinio-6-yl)amido]-6-[2(-1-methylpiperidinio-1-yl)ethylamido]-2,6-pyridinedicarboxylic acid diiodide
   this product of formula (I) may be in all the possible isomeric forms, such as the racemic, enantiomeric and diastereoisomeric forms, and the addition salts with inorganic or organic acids or with inorganic or organic bases

The subject of the present invention is also a method for preparing the products of formula (IB) according to the present invention: a general method of synthesis is thus described as follows.

The subject of the present invention is also a method for preparing the products of formula (IB) according to the present invention: a general method of synthesis is thus described as follows.

General Method of Synthesis

A particularly advantageous method in the context of the invention consists in alkylating, at the end of the synthesis, a product of general formula (IA) to a product of general formula (IB) with the aid of an alkyl halide, or optionally of an alkyl sulfate according to the general scheme below:

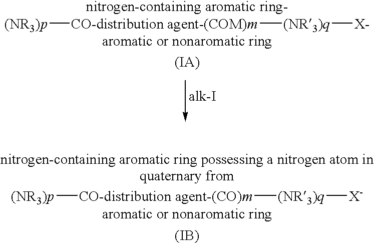

The products of general formula (IA) in which X represents a single bond may be prepared according to any of the general methods of synthesis described in patent WO 0296903.

The products of general formula (IA) in which X is different from a single bond may be advantageously prepared according to the general scheme below:

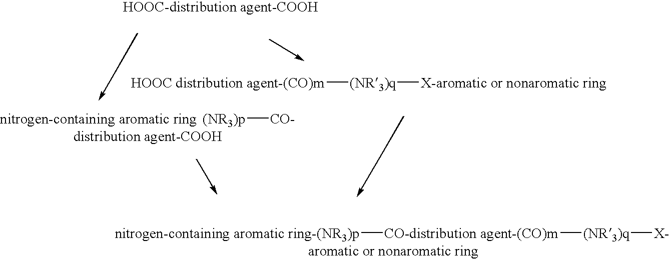

The subject of the present invention is thus the products of formula (IB) as defined above, characterized in that they have a telomerase inhibiting activity.

The subject of the present invention is thus the products of the formula (IB) as defined above, characterized in that they have an anticancer activity.

The subject of the present invention is also, as medicaments, the products of formula (IB) as defined above, and their prodrugs, said products of formula (IB) may be in all the possible isomeric forms, such as the racemic, enantiomeric and diastereoisomeric forms, and the addition salts with pharmaceutically acceptable inorganic or organic acids or with inorganic or organic bases of said products of formula (IB).

The subject of the present invention is thus, as medicaments, the products of formula (Ia) as defined in the preceding claims, and their prodrugs, said products of formula (Ia) may be in all the possible isomeric forms, such as the racemic, enantiomeric and diastereoisomeric forms, and the addition salts with pharmaceutically acceptable inorganic or organic acids or with inorganic or organic bases of said products of formula (Ia).

The subject of the present invention is particularly, as medicaments, the products described below in the experimental part, and their prodrugs, and the addition salts with pharmaceutically acceptable inorganic or organic acids or inorganic or organic bases of these products.

The term patient denotes human beings, but also other mammals.

The term “Prodrug” denotes a product which may be converted in vivo by metabolic mechanisms (such as hydrolysis) to a product of formula (IB). For example, an ester of a product of formula (IB) containing a hydroxyl group may be converted by hydrolysis in vivo to its parent molecule. Alternatively, an ester of a product of formula (IB) containing a carboxyl group may be converted by hydrolysis in vivo to its parent molecule.

The products may be administered parenterally, orally, perlingually, rectally or topically.

The subject of the invention is also the pharmaceutical compositions, characterized in that they contain, as active ingredient, one or more medicaments of general formula (IB) as defined above and in particular the products described below in the experimental part.

These compounds may be provided in the form of solutions or suspensions for injection, tablets, coated tablets, capsules, syrups, suppositories, creams, ointments and lotions. These pharmaceutical dosage forms are prepared according to the customary methods. The active ingredient may be incorporated into excipients normally used in these compositions, such as aqueous or nonaqueous vehicles, talc, gum arabic, lactose, starch, magnesium stearate, cocoa butter, fat of animal or plant origin, paraffin derivatives, glycol, various wetting, dispersing or emulsifying agents, and preservatives.

The customary dose, which can vary according to the subject treated and the condition in question, may be, for example, from 10 mg to 500 mg per day in humans, by the oral route.

The subject of the present invention is also the pharmaceutical compositions as defined above, containing, in addition, active ingredients of other chemotherapy medicaments against cancer.

The subject of the present invention is also the pharmaceutical compositions as defined above, characterized in that they are used as medicaments, in particular for cancer chemotherapy.

The subject of the present invention is thus the use of the compounds defined above as a pharmaceutical product for human use.

The present invention also relates to the therapeutic combinations consisting of a compound of formula (IB) as defined above and another anticancer compound.

The present invention thus relates to therapeutic combinations as defined above, characterized in that the anticancer compound is chosen from alkylating agents, platinum derivatives, antibiotics, antimicrotubule agents, anthracyclines, groups I and II topoisomerases, fluoropyrimidines, cytidine analogs, adenosine analogs, various enzymes and compounds such as L-asparaginase, hydroxyurea, trans-retinoic acid, suramin, irinotecan, topotecan, dexrazoxane, amifostine, herceptin and the estrogen and androgen hormones, and antivascular agents.

The present invention also relates to a therapeutic combination consisting of a compound of formula (IB) as defined above and radiation.

The present invention thus relates to the combinations as defined above, characterized in that each of the compounds or of the treatments is administered simultaneously, separately or sequentially.

The present invention thus relates to the use of products of formula (IB) as defined above or of pharmaceutically acceptable salts of said products of formula (IB) for the preparation of medicaments for treating cancers, genetic diseases or pilosity abnormalities.

The present invention thus relates to the use of products of formula (IB) as defined above or of pharmaceutically acceptable salts of said products of formula (I) for the preparation of a medicament for treating cancer.

The present invention thus relates to the use of products of formula (IB) as defined above or of pharmaceutically acceptable salts of said products of formula (I) for the preparation of a medicament intended for treating genetic diseases such as Bloom's syndrome, Werner's syndrome, Rothmund-Thomson syndrome or ataxia telangiectasia syndrome.

The present invention thus relates to the use of products of formula (IB) as defined above or of pharmaceutically acceptable salts of said products of formula (IB) for the preparation of a medicament intended for treating pilosity abnormalities such as hyperpilosity.

The present invention particularly relates to the use of products of formula (IB) as defined above or of pharmaceutically acceptable salts of said products of formula (IB) for the preparation of a medicament intended for treating cancer of the breast, stomach, colon, lungs, ovaries, uterus, brain, kidney, larynx, lymphatic system, thyroid, urogenital tract, tract including vesicle and prostate, bones and pancreas, melanomas and more particularly cancer of the breast, colon or lungs.

The present invention relates particularly to the use of products of formula (IB) as defined above or of pharmaceutically acceptable salts of said products of formula (IB) for the preparation of a medicament intended for cancer chemotherapy and in particular intended for cancer chemotherapy used alone or in combination.

The present invention thus relates to the use of products of formula (IB) as defined above in any one of the preceding claims or of pharmaceutically acceptable salts of said products of formula (IB) for the preparation of medicaments intended to be used alone or in combination with chemotherapy or radiotherapy or alternatively in combination with other therapeutic agents, and in particular such a use in which the therapeutic agents may be commonly used antitumor agents.

The products of formula (IB) according to the present invention may thus also be advantageously used in combination with antiproliferative agents: by way of examples of such antiproliferative agents but without this list being however limiting, there may be mentioned aromatase inhibitors, antiestrogens, topoisomerase I inhibitors, topoisomerase II inhibitors, agents which are active on the microtubules, alkylating agents, histone deacetylase inhibitors, farnesyl transferase inhibitors, COX-2 inhibitors, MMP inhibitors, mTOR inhibitors, antineoplastic antimetabolites, platinum compounds, compounds which decrease the activity of protein kinases and also antiangiogenic compounds, gonadorelin agonists, antiandrogens, bengamides, biphosphonates and trastuzumab.

There may thus be mentioned, by way of examples, antimicrotubule agents such as taxoids, vinca alkaloids, alkylating agents such as cyclophosphamide, DNA-intercalating agents such as cis-platinum, agents which interact with topoisomerase such as camptothecin and derivatives, anthracyclines such as adriamycin, antimetabolites such as 5-fluorouracil and derivatives and analogs.

The affinity and the selectivity of the products of general formula (IB) according to the present invention for G-quadruplex DNA structures may be determined by one or more of the following methods:

Test of affinity No. 1: Measurement of the inhibition of pairinq of an oligonucleotide, capable of forming a G-guadruplex structure, with its complementary strand measured in the form of a 50% inhibitory concentration IC50, expressed in μM, by a luminescence method according to the experimental protocol is described below.

The principle of this test uses the activation of “acceptor” beads by an oxygen singlet emitted by “donor” beads excited by laser when the “acceptor” and “donor” beads are in proximity. This test was developed by Packard Bioscience under the name Amplified Luminescent Proximity Homogeneous Assay or ALPHA screen.

The “donor” beads are conjugated with streptavidin and the acceptor beads with an anti-digoxigenin antibody (catalog reference 6760604). A DNA strand, in this case the telomeric strand, is coupled at its 5′ end to biotin so as to be able to bind to the “donor” beads, while the complementary strand is coupled to digoxigenin so as to be able to bind to the “acceptor” beads. During the pairing of the telomeric strand with its complementary strand, the beads are placed in proximity and a luminescence signal is then emitted at a wavelength of 520-620 nm.

The oligonucleotides used in the experiments were synthesized by Perkin Elmer Life Sciences (Finland). The G-rich strand corresponding to the repetitive units of human telomeric DNA possess the sequence G-GTT-TAA-AAT-AAT-TGA-GGG-TTA-GGG-TTA-GGG-TTA-GGG. The complementary strand has the sequence GGT-TTA-AAA-AAT-TTG-CCC-TAA-CCC-TAA-CCC-TAA-CCC-T. The biotin or the digoxigenin are added to the 5′ end of the oligonucleotides.

The experiments are carried out in 50 mM TRIS-HCl buffer pH 7.4 containing 100 mM KCl and 0.1% BSA.

The measurements are carried out with an Alphaquest microplate analyzer (Fusion α) from Packard Biosciences.

The experiments are carried out in 96-well plates (½ wells). An oligonucleotide stock solution at the concentration of 25 nM in the buffer described above is prepared. 10 μl of this solution are distributed into the wells. 10 μl of the test product at different concentrations, prepared in the same buffer containing 0.6% DMSO, are then added. 10 μl of buffer+0.6% DMSO are distributed into the control wells. The samples are left to incubate for 15 minutes at room temperature. After this incubation time, 10 μl of the complementary strand at the concentration of 25 nM and 20 μl of a solution containing 2 types of bead diluted beforehand to 50 mg/ml are added to the wells. The plates are incubated at room temperature for 2 hours before reading. Under these conditions, the telomeric strand may adopt a secondary conformation of the G-quadruplex type which the product, depending on its affinity for this structure, stabilizes by preventing pairing with the complementary strand. The signal emitted is then minimal. In the absence of products in the control wells, the telomeric strand pairs with the complementary strand, resulting in a maximum signal.

Test of selectivity No. 1: Measurement of the inhibition of pairing of any oligonucleotide with its complementary strand measured in the form of a 50% inhibitory concentration IC50, expressed in μM, by a luminescence method according to the experimental protocol is described below.

The test used is the same as that described above. Only the oligonucleotides are different, the telomeric strand being replaced by an oligonucleotide having the following sequence: G-GTT-TAA-AAT-AAT-TGA-GGC-TTA-CCG-TTA-CCG-TTA-CGG biotinylated at the 5′ end. The complementary strand has the sequence: 5′-GGT-TTA-AAA-AAT-TTG-CGG-TAA-CGG-TAA-CGG-TAA-GCC-T labeled with digoxigenin at the 5′ end. If the test product has affinity for the biotinylated DNA sequence, the pairing of the complementary strand will be prevented and the signal obtained will be minimal. In the absence of product or if the latter has no affinity for this DNA, the pairing will occur and the signal will be maximum.

Test of affinity No. 2: Measurement of the dissociation constant, expressed in μM, of the complex between a product of the invention and an oligonucleotide, capable of forming a monomeric G-guadruplex structure, by a fluorescence method according to the experimental protocol is described below.

The titrations are carried out at 20° C. in a quartz cuvette having a useful volume of 3 ml and a cross section of 10×10 mm, placed in the thermostated compartment of the Spex Fluorolog 3 fluorimeter (Jobin-Yvon). The buffer used in all the experiments is sodium cacodylate, pH 7.2 (10 mM) containing 100 mM potassium chloride. To a 0.1 μM solution of compound are added increasing concentrations of nucleic acids. After an equilibration time of 3 minutes, a fluorescence emission spectrum is recorded for each point, using slits of 5 nm and an excitation wavelength of 340 nm. Each aliquot represents an additional volume of 3 microliters. The effects of dilution are corrected at the end of the experiment after integrating the emission signal. The curves representing the emission intensity as a function of the nucleic acid concentration are then analyzed and fitted with the Kaleidagraph 3.52 software for Macintosh.

Test of selectivity No. 2: Measurement of the dissociation constant, expressed in μM, of the complex between a product of the invention and any double-stranded oligonucleotide, by a fluorescence method according to the experimental protocol is described below.

The titrations are carried out according to a protocol identical to that used for the titrations of the preceding test.

Test of affinity No. 3: Measurement of the stabilization of the G-guadruplexes Tm, expressed in ° C., by a method using the formation of a complex with fluorescein of which the experimental protocol is described below.

Oligonucleotides

All the oligonucleotides, modified or otherwise, were synthesized by Eurogentec S A, Seraing, Belgium. The oligonucleotide FAM+DABCYL has the catalog reference OL-0371-0802. It has the sequence: GGGTTAGGGTTAGGGTTAGGG corresponding to 3.5 repeats of the human telomeric unit (strand rich in G). The fluorescein is attached to the 5′ end, the DABCYL to the 3′ end, by the chemical arms described by Eurogentec. An oligonucleotide FAM+TAMRA may also be used. The concentration of the samples is checked by spectrophotometry, by recording the absorbance spectrum between 220 and 700 nm and using the molar extinction coefficient provided by the supplier.

Buffers

All the experiments were carried out in a 10 mM sodium cacodylate buffer pH 7.6 containing 0.1 M lithium chloride (or sodium chloride). The absence of fluorescent contamination in the buffer was checked beforehand. The fluorescent oligonucleotide is added at the final concentration of 0.2 μM.

Study of Fluorescence

All the measurements of fluorescence were carried out on a Spex Fluorolog DM1B or Fluoromax 3 apparatus, using an excitation line width of 1.8 nm and an emission line width of 4.5 or 5 nm. The samples are placed in a microquartz cuvette of 0.2×1 cm. The temperature of the sample is controlled by an external water bath. The oligonucleotide alone was analyzed at 20, 30, 40, 50, 60, 70 and 80° C. The emission spectra are recorded using an excitation wavelength of 470 nm. The excitation spectra are recorded using either 515 or 588 nm as emission wavelength. The spectra are corrected for the response of the instrument by reference curves. A high extinction (80-90%) of the fluorescence of fluorescein at room temperature is observed, in agreement with an intramolecular folding of the oligonucleotide at 20° C. in the form of a G-quadruplex, which induces juxtaposition of its 5′ and 3′ ends which are respectively linked to fluorescein and to DABCYL. This juxtaposition causes an already-described phenomenon of extinction of fluorescence which is used for “Molecular Beacons”.

Fluorescence Tm

An oligonucleotide stock solution at the strand concentration of 0.2 μM in 0.1 M LiCl, 10 mM cacodylate buffer, pH 7.6, is prepared beforehand, heated briefly at 90° C. and slowly cooled to 20° C., and then distributed in aliquots of 600 μl into the fluorescence cuvettes. 3 μl of water (for the control) or 3 μl of test product (stock at 200 μM, final concentration 1 μM) are then added and mixed. The samples are then allowed to incubate for at least 1 hour at 20° C. before each measurement. The use of longer incubation times (up to 24 hours) has no influence of the result obtained. Each experiment allows the measurement of 1 to 4 samples. They are first incubated at an initial temperature of 20° C. and heated to 80° C. over 38 minutes. During this time, the fluorescence is measured simultaneously at one emission wavelength (515 nm) or at two emission wavelengths (515 nm and 588 nm) using 470 nm as excitation wavelength. A measurement is carried out every 30 seconds or every degree. The temperature of the water bath is recorded in parallel, and the fluorescence profile as a function of the temperature is reconstituted from these values. The fluorescence profiles are then normalized between 20° C. and 80° C., and the temperature for which the intensity of emission at 515 nm is the mean of those at high or low temperature is called Tm. Under these conditions, the Tm of the reference sample without addition of product is 44° C. in a lithium chloride buffer. This temperature is increased to more than 55° C. in a sodium chloride buffer. The addition of a G-quadruplex stabilizing compound induces an increase in the Tm. This increase is judged to be significant if it is greater than 3°.

Test of selectivity No. 3: Estimation of the distribution of equilibrium of a product of the invention between various oligonucleotides or DNA structures, by a dialysis method according to the experimental protocol is described below.

All the polynucleotides are obtained from Amersham-Pharmacia. The oligonucleotides were synthesized by Eurogentec, Belgium on the scale of 1 μmol and used without further purification. 19 structures are tested in parallel (sample numbered from 1-19, see table below). The triplexes TC, GA and GT result from the combination of two strands of different lengths (13 and 30 bases):

5′ GAAAGAGAGGAGG and 5′ CCTCCTCTCTTTCCCTTCTTTCTCTCCTCC (TC triplex, sample #1);

5′ CCTCCTCTCTTTC and 5′ GAAAGAGAGGAGGCCTTGGAGGAGAGAAAG (GA triplex, sample #2);

5′ CCTCCTCTCTTTC and 5′ GAAAGAGAGGAGGCCTTGGTGGTGTGTTTG (GT triplex, sample #3).

The “duplex” GA (sample #5) results from the self-pairing of the oligonucleotide (5′ GAGAGAGAGAGAGAGAGAGAGAGA). The parallel duplex (sample #6) results from the combination of 5′ AAAAAAAAAATAATTTTAAATATT with 5′ TTTTTTTTTTATTAAAATTTATAA. 24 CTG (sample #7) mimics 8 repeats of trinucleotide: 5′ CTGCTGCTGCTGCTGCTGCTGCTG. ds26 (sample #11) is a self-complementary duplex of 26 bases 5′ CAATCGGATCGAATTCGATCCGATTG. 22CT (sample #13) is an oligonucleotide which mimics the C-rich strand of human telomeres: 5′ CCCTAACCCTAACCCTAACCCTAACCCT, while 22AG (sample #14) is an oligonucleotide which mimics the G-rich strand of human telomeres: 5′ AGGGTTAGGGTTAGGGTTAGGG. 24G20 (T₂G₂₀T₂, sample #15) can form an intermolecular quadruplex 5′ (TTGGGGGGGGGGGGGGGGGGGGTT)₄.

Table of the structures of the nucleic acids used in dialysis

| Number | Name | Type^{a} (length) | Structure | Tm (° C.) |
|---|---|---|---|---|
| 1 | triplex TC | oligos (30 + 13) | Triplex | 38 |
| 2 | triplex GA | oligos (30 + 13) | Triplex | 53 |
| 3 | GT triplex | oligos (30 + 13) | Triplex | 53 |
| 4 | poly dA.2polydT | poly | Triplex | 71 |
| 5 | duplex GA | oligo (24) | “Duplex”^{b} | 37 |
| 6 | parallel duplex | oligos (30 + 30) | “Duplex”^{b} | 39 |
| 7 | 24CTG | oligo (24) | “Duplex”^{b} | 64 |
| 8 | poly d(A-T) | poly | Duplex | 66 |
| 9 | poly d(G-C) | poly | Duplex | >90 |
| 10 | CT DNA | poly | Duplex | 86 |
| 11 | ds 26 | oligo (26) | Duplex | 75 |
| 12 | poly dC | poly | i-DNA | 51 |
| 13 | 22CT | oligo (22) | ss/i-DNA | 13 |
| 14 | 22AG | oligo (22) | G4 | 62 |
| 15 | 24G20 | oligo (24) | G4 | >90 |
| 16 | poly dT | poly | single strand | — |
| 17 | poly dA | poly | single strand | — |
| 18 | poly rU | poly | single strand | — |
| 19 | poly rA | poly | single strand | — |

^{a}poly = polynucleotide; oligo = oligonucleotide; oligos = structure formed by the combination of two different oligonucleotides. The lengths are indicated in brackets. The polynucleotides are more than 100 bases long.
^{b}These duplexes involve the formation of noncanonic base pairs.

Antitelomerase activity of the products of the invention, specifically dependent on the stabilization of the G-quadruplex structure, measured by the 50% inhibitory concentration IC50 expressed in μM, can be evaluated according to the protocol below:

Preparation of the Extract Enriched with Human Telomerase Activity

The pulmonary carcinoma line A549 is obtained from ATCC (American Type Culture Collection, Rockville USA). The cells are cultured in a layer, in a culture flask in DMEM medium, supplemented with 2 mM glutamax, 200 U/ml penicillin, 200 μg/ml streptomycin and 10% heat-inactivated fetal calf serum. The cells at the exponential growth phase are trypsinized, washed in 1× PBS and an aliquot of 10⁶ cells is centrifuged at 3000 xg and the supernatent removed. The cell pellet is resuspended by several successive pipettings in 200 μl of lysis buffer containing 0.5% CHAPS, 10 mM Tris-HCl pH 7.5, 1 mM MgCl₂, 1 mM EGTA, 5 mM β-mercaptoethanol, 0.1 mM PMSF and 10% glycerol and is stored on ice for 30 minutes. The lysate is centrifuged at 160 000 xg for 20 minutes at 4° C. and 160 μl of supernatant is recovered. The proteins of the extract are assayed by the Bradford method. The extract is stored at −80° C.

Assay of the Telomerase Activity by the Trap-G4 Assay

The inhibition of the telomerase activity is determined by a modified TRAP protocol which makes it possible to measure the telomerase extension using an oligonucleotide TSG4 (^{5′}GGGATTGGGATTGGGATTGGGTT^{3′}) which can form an intramolecular G-quadruplex structure, in the presence of a cellular extract enriched with telomerase activity and compounds which are added at various concentrations (30, 10, 1, 0.1 and 0.01 μM). The extension reaction is followed by a PCR amplification of the extension products with the aid of the oligonucleotide CXext (^{5′}GTGCCCTTACCCTTACCCTTACCCTAA^{3′}). The selectivity of the inhibition is measured by the amplification of a control oligonucleotide TSNT (^{5′}ATTCCGTCGAGCAGAGTTAAAAGGCCGAGAAGCGAT^{3′}) by the oligonucleotide TS (^{5′}AATCGTTCGAGCAGAGTT^{3′}) and the oligonucleotide NT (^{5′}ATCGCTTCTCGGCCTTTT^{3′}).

The reaction medium is prepared in a final volume of 50 μl based on the following composition:

| Tris HCl pH 8.0 | 20 | mM |
|---|---|---|
| MgCl2 | 1.5 | mM |
| KCl | 63 | mM |
| Tween 20 | 0.005% (W/V) | |
| EGTA | 1 | mM |
| dATP | 50 | μM |
| dGTP | 50 | μM |
| dCTP | 50 | μM |
| dTTP | 50 | μM |
| Oligonucleotide TSG4 | 3.5 | picomol |
| Oligonucleotide CXext | 22.5 | picomol |
| Oligonucleotide TSNT | 0.01 | attomol |
| Oligonucleotide NT | 7.5 | picomol |
| Oligonucleotide TS | 18 | picomol |
| Bovine serum albumin | 20 | μg/ml |
| Taq DNA polymerase | 50 | U/ml |
| Telomerase extract | 100 ng in a volume of 1 μl | |
| Test product or solvent | in a volume of 5 μl | |
| Double-distilled water qs | 50 | μl |

The oligonucleotides are obtained from Eurogentec Belgium and are stored at −20° C. at a stock concentration of 100 μM in sterile distilled water without ribonucleases and deoxyribonucleases.

The reaction samples are combined on ice in 0.2 ml PCR tubes.

The reaction samples are then incubated in an Eppendorf Mastercycler PCR apparatus based on the following temperature conditions:
15 minutes at 30° C.,
1 minute at 90° C.,
followed by 30 cycles of
30 seconds at 92° C.,
30 seconds at 52° C.,
30 seconds at 72° C.,
followed by a final cycle of 2 minutes at 72° C.

After the amplification, 8 μl of a loading buffer having the following composition are added to the samples:

| | |
|---|---|
| TBE | 5× |
| sucrose | 20% (W/V) |
| Bromophenol blue | 0.2% |
| Xylene cyanol | 0.2% |

The samples are then analyzed by electrophoresis on a 12% acrylamide/bisacrylamide (19:1) gel in TBE 1× buffer for 45 minutes at a voltage of 200 volts, with the aid of a Novex electrophoresis system.

The gels are stained for 15 minutes in a 1× solution of SYBR Green (Roche) and the fluorescence of the PCR products is digitized by a digital camera (Bioprint system).

The disappearance of the band formed by the dimerization of the oligonucleotides TSG4 and CXext corresponds to a stabilization of the G-quadruplex form of the oligonucleotide TSG4 and corresponds to an inhibition of the extension of the telomeric repeats from the oligonucleotide TSG4.

The disappearance of the band formed by the amplification of the control oligonucleotide TSNT corresponds to a nonspecific inhibition of the activity of Taq polymerase.

For each compound, the results are expressed by calculating the concentration (μM) inhibiting 50% of the formation of the TSG4-Cxext band (IC50 TRAP-G4) and by calculating the concentration inhibiting 50% of the formation of the TSNT control band (IC50 Taq), compared with the value for the enzyme sample with no compound.

The IC50 Taq/IC50 TRAP-G4 ratio indicates the selectivity factor for the inhibition of the telomerase extension by G-quadruplex stabilization compared with the Taq polymerase inhibition.

It is considered that a compound is active as antitelomerase agent stabilizing the G-quadruplex DNA when the IC50 TRAP-G4 is in particular less than 5 μM.

It is considered that the compound is selective as antitelomerase agent stabilizing the G-quadruplex when the IC50 TRAP-G4/IC50 Taq ratio is greater than 3.

Table of biological results

| | Affinity/selectivity G4 | | Affinity/selectivity G4 | | Affinity G4 | Selectivity G4 | | |
|---|---|---|---|---|---|---|---|---|
| | Test 1 (alpha screen) | | Test 2 (Kd by fluorescence) | | Test 3 | Test 3 | Inhibition of telomerase | |
| | Affinity | Selectivity | | Selectivity | Delta | Dialysis at | | Selectivity |
| Example | IC50 μM | factor | Kd μM | factor | Tm ° C. | equilibrium | IC50 μM | factor |
| 1 | 0.16 | >20 | 0.007 | >>100 | 26 | see FIG. 3 | 0.45 | 33 |
| | cf FIG. 1 | cf FIG. 1 | cf FIG. 2 | cf FIG. 2 | | | | |
| 2 | 0.03 | 34.7 | nd | nd | 24.5 | nd | 0.3 | 70 |
| | | | | | | | cf FIG. 4 | cf FIG. 4 |
| 3 | 0.13 | >25 | nd | nd | 19 | nd | 1 | 40 |
| 4 | 0.24 | >20 | nd | nd | 6.2 | nd | 3 | >10 |
| 5 | 0.1 | >30 | nd | nd | 17 | nd | 0.45 | 35 |
| 6 | 0.02 | 10 | nd | nd | 23.7 | nd | 0.3 | 10 |
| 7 | 0.52 | >5 | nd | nd | 9.1 | nd | 3 | >3.3 |
| 8 | 0.55 | >5 | nd | nd | 6.2 | nd | 3 | >3.3 |
| 9 | 0.06 | >50 | 0.0007 (K+) | >1000 | 21 | cf FIG. 3 | 0.3 | 150 |
| | | | 0.0032 (Na+). | cf FIG. 2 | | | cf FIG. 4 | cf FIG. 4 |
| | | | cf FIG. 2 | | | | | |
| 10 | 0.78 | >5 | nd | nd | 11.9 | cf FIG. 3 | 1 | >10 |
| 11 | 0.05 | >60 | nd | nd | 21.8 | nd | 0.6 | 116 |
| 12 | 1.06 | >3 | nd | nd | 12.1 | nd | 1.5 | >66 |
| 13 | 1.27 | >3 | nd | nd | nd | nd | nd | Nd |
| 14 | 0.6 | >5 | nd | nd | nd | nd | nd | nd |
| 15 | 0.02 | 64 | nd | nd | nd | nd | nd | nd |

### BRIEF DESCRIPTION OF THE DRAWINGS

FIG. 1 shows results of Test 1, i.e. the affinity (curve G4/Com) and selectivity (curve C+/C−) of the compounds of the invention by measuring the inhibition of pairing of oligonucleotides with their complementary strands in a bioluminescence test.

FIG. 2 illustrates Test 2, i.e. the determination of the dissociation constant of the complex between the products of the invention and, on the one hand, an oligonucleotide G4 (affinity) and, on the other hand, a DNA double strand (selectivity).

FIGS. 3 and 3′(Test of selectivity) shows the estimation of distribution at equilibrium of the instant invention between various oligonucleotides or DNA structures.

FIG. 4 Illustrates the determination of the antitelomerase activity of the products of the invention, specifically dependent on the stabilization of the G-quadruplex structure.

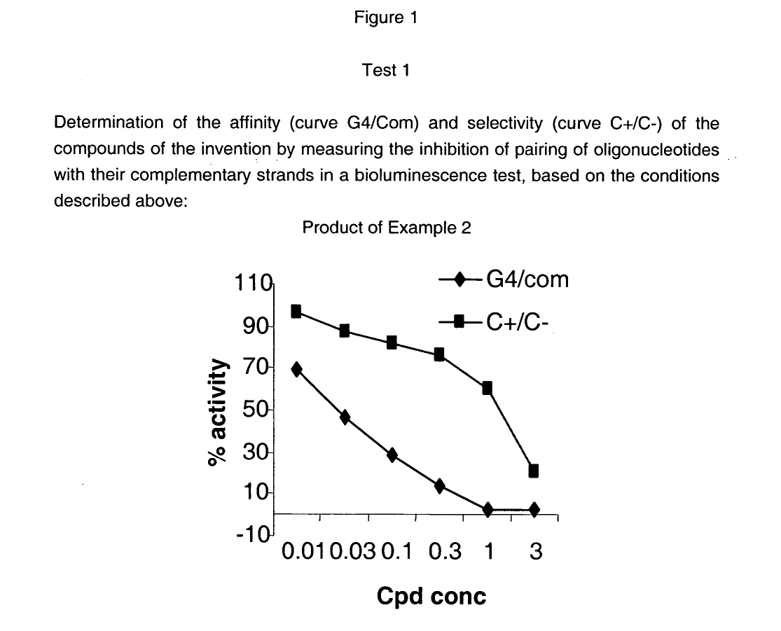

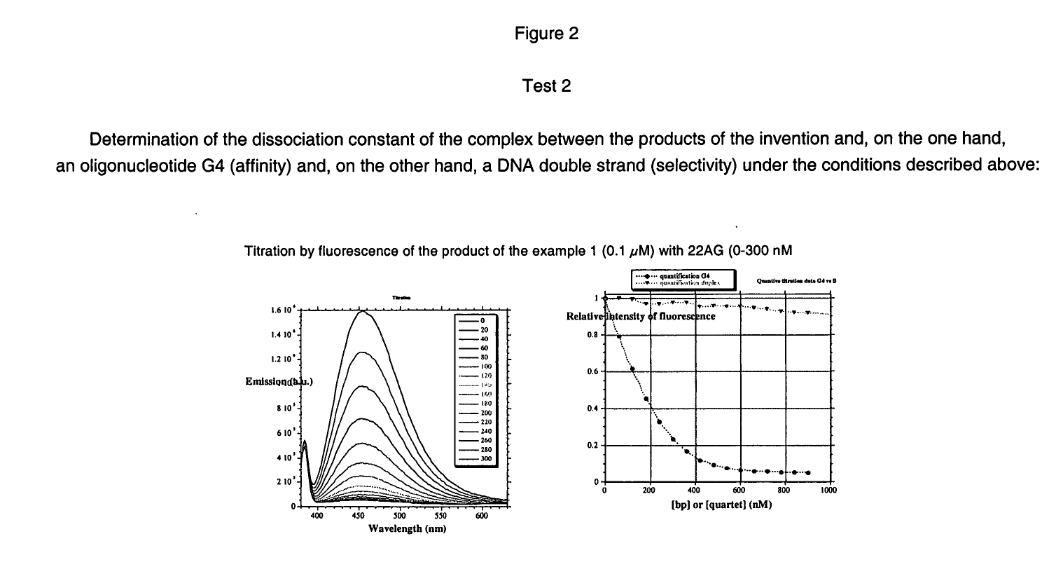

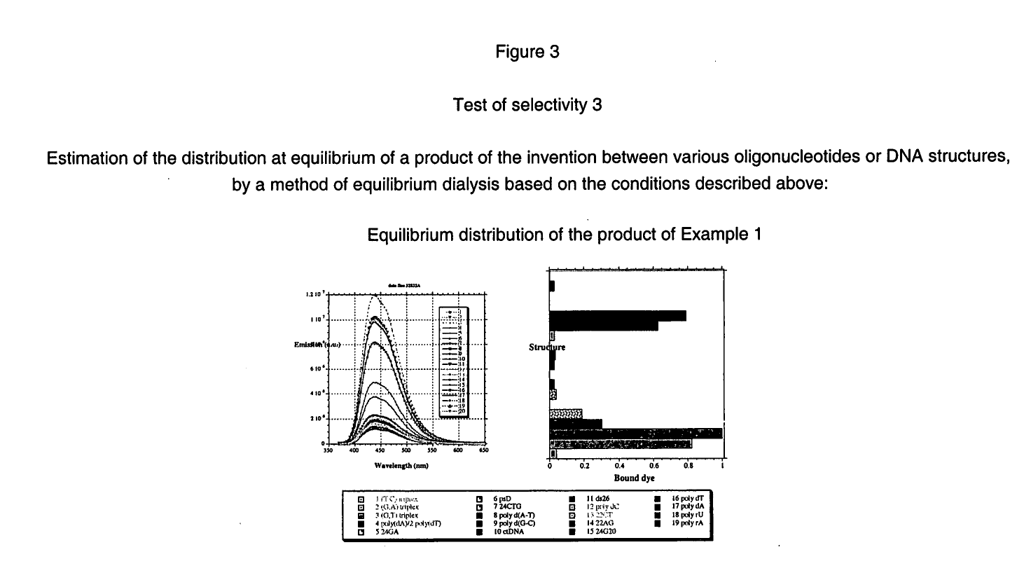

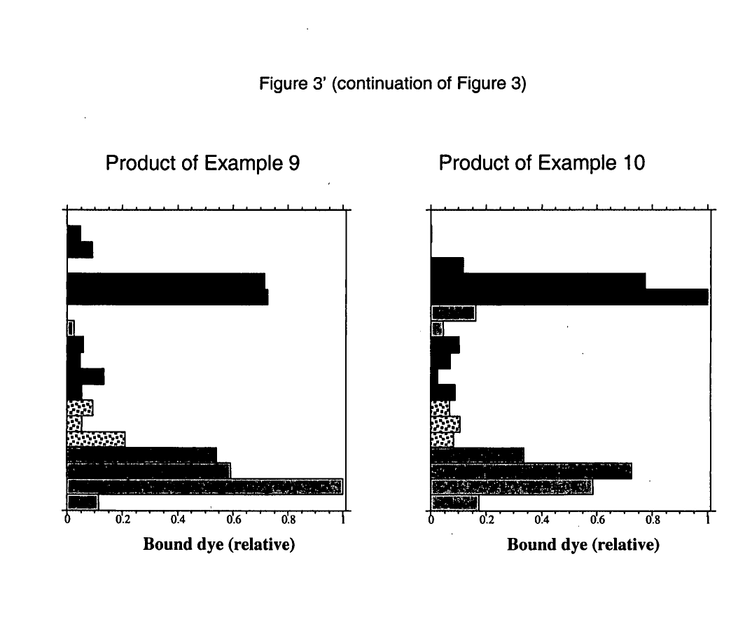

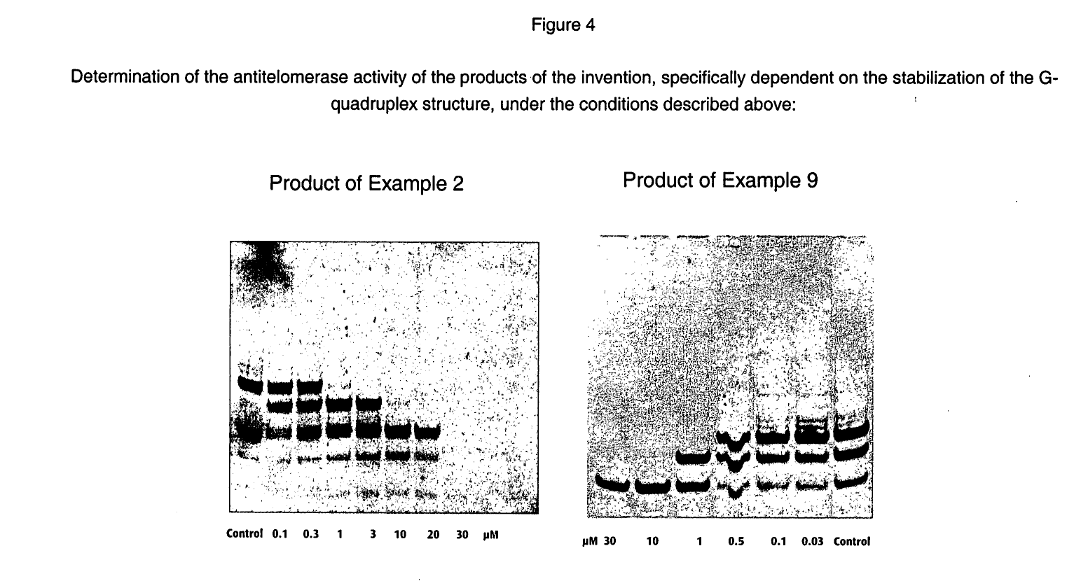

The following and nonlimiting examples are given to illustrate the invention.

### EXAMPLE 1

### Preparation of bis[(1-methylquinolinio-6-yl)amido]-2,6-pyridinedicarboxylic acid diiodide

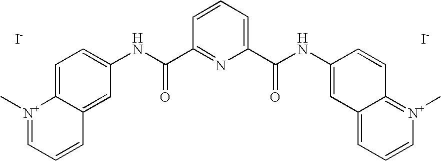

Step 1: 1 g of 2,6-pyridinedicarboxylic acid and 1.81 g of 6-aminoquinoline are dissolved, in a 50 ml three-necked round-bottomed flask under magnetic stirring, in 30 ml of dichloromethane and 5 ml of dimethylformamide (DMF), and then 2.4 g of 1-(3-dimethylaminopropyl)carbodiimide hydrochloride (EDCI) and 162 mg of 1-hydroxybenzotriazole (HOBT) are added. A transient yellow precipitate forms which slowly redissolves. After stirring for 1 to 2 hours at room temperature, an abundant white precipitate appears. After stirring overnight at room temperature, the reaction is complete (checked by liquid chromatography coupled to mass spectroscopy LC/MS). The precipitate formed is drained, washed successively with dichloromethane and water, and then dried under reduced pressure in the presence of phosphoric anhydride. 2.41 g of bis[(quinolin-6-yl)amido]-2,6-pyridinedicarboxylic acid are thus obtained in the form of a white powder which is used as it is in the next step.

Step 2: 200 mg of bis[(quinolin-6-yl)amido]-2,6-pyridinedicarboxylic acid, obtained in the preceding step, are dissolved in a 25 ml round-bottomed flask, in 2 ml of methanol, 4 ml of DMF and 10 ml of iodomethane, and then the medium is heated at 500 for 72 hours during which an orange precipitate gradually forms. After cooling, this precipitate is drained and washed with methanol. After recrystallization from a mixture of ethanol and DMF (50/50 by volume), 288 mg of bis[(1-methylquinolinio-6-yl)amido]-2,6-pyridinedicarboxylic acid diiodide are obtained in the form of orange-yellow crystals whose characteristics are the following:

Melting point (Kofler)>260° C.

¹H NMR spectrum (300 MHz, (CD₃)₂SO d6, δ in ppm): 4.70 (broad s: 6H); from 8.15 to 8.30 (mt: 2H); from 8.40 to 8.80 (mt: 7H); 9.25 (d, J=2 Hz: 2H); 9.37 (broad d, J=8.5 Hz: 2H); 9.45 (broad d, J=5.5 Hz: 2H); 11.64 (broad s: 2H).

### EXAMPLE 2

### Preparation of 2-[(1-methylquinolinio-6-yl)amido]-6-[(4-dimethylamino-1-methylquinaldinio-6-yl)amido]-2,6-pyridinedicarboxylic acid diiodide

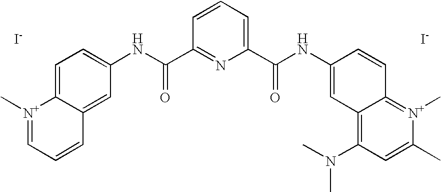

Step 1: 1.3 g of n-butyl monoester of 2,6-pyridinedicarboxylic acid, which may be obtained according to Khim. Geterosikl. Soedin 1976(2), 233-7, and 1.71 g of 6-amino-4-dimethylaminoquinaldine, which may be obtained according to WO 01/40218, are dissolved, in a 50 ml three-necked round-bottomed flask, in 35 ml of dichloromethane, and then 1.35 g of EDCI and 90 mg of HOBT are added. After 2 to 3 hours, a yellow precipitate appears; the stirring is maintained for 36 hours at room temperature, until the reaction is complete (LC/MS). The reaction medium is diluted with water, the organic phase is separated after settling and the aqueous phase is extracted with dichloromethane. The combined organic phases are concentrated to dryness under reduced pressure. The pasty residue obtained is taken up, with stirring, in 20 ml of diisopropyl ether, to form a light beige solid which is drained and air-dried. 820 mg of n-butyl ester of 6-[(4-dimethylaminoquinaldin-6-yl)amido]-2,6-pyridinedicarboxylic acid, which is used as it is in the next step, are thus obtained.

Step 2: In a 50 ml round-bottomed flask, a solution of 820 mg of n-butyl ester of 6-[(4-dimethylaminoquinaidin-6-yl)amido]-2,6-pyridinedicarboxylic acid, obtained above, in 30 ml of n-butanol is stirred for 16 hours with 2 ml of a 1 M aqueous potassium hydroxide solution. After concentrating under reduced pressure, the residue is taken up in 10 ml of water and neutralized to pH=6 by adding a 0.1 M aqueous hydrochloric acid solution. The precipitate formed is drained, washed with water and dried under reduced pressure at 600 in the presence of phosphoric anhydride. 760 mg of 6-[(4-dimethylaminoquinaldin-6-yl)amido]-2,6-pyridinedicarboxylic acid are thus obtained in the form of a white solid which is used as it is in the next step.

Step 3: 760 mg 6-[(4-dimethylaminoquinaldin-6-yl)amido]-2,6-pyridinedicarboxylic acid, obtained above, and 360 mg of 6-aminoquinoline are dissolved, in a 50 ml round-bottomed flask, in 15 ml of DMF, and then 458 mg of EDCI and 30 mg of HOBT are added. After stirring for 72 hours at room temperature, the solvent is removed under reduced pressure. The residue is taken up in water, and the precipitate thus formed is washed with water and then with a saturated sodium hydrogen carbonate solution. After drying under reduced pressure in the presence of phosphoric anhydride, 852 mg of 2-[(quinolin-6-yl)amido]-6-[(4-dimethylaminoquinaldin-6-yl)amido]-2,6-pyridinedicarboxylic acid are obtained in the form of a beige powder which is used as it is in the next step.

Step 4: The procedure is carried out as in Step 2 of Example 1, but starting with 400 mg of 2-[(quinolin-6-yl)amido]-6-[(4-dimethylaminoquinaldin-6-yl)amido]-2,6-pyridinedicarboxylic acid, obtained above in 6 ml of methanol and 15 ml of iodomethane. 382 mg of 2-[(1-methylquinolinio-6-yl)amido]-6-[(4-dimethylamino-1-methylquinaldinio-6-yl)amido]-2,6-pyridinedicarboxylic acid diiodide are thus obtained, after recrystallization from ethanol, in the form of a pale green solid whose characteristics are the following:

Melting point (Kofler)>260° C.

¹H NMR spectrum (300 MHz, (CD₃)₂SO d6, δ in ppm): 2.85 (broad s: 3H); 3.56 (s: 6H); 4.11 (broad s: 3H); 4.71 (broad s: 3H); 7.12 (broad s: 1H); 8.20 (broad dd, J=8.5 and 5.5 Hz: 1H); from 8.30 to 8.80 (mt: 7H); 9.03 and 9.05 (2 broad s: 2H in total); 9.36 (broad d, J=8.5 Hz: 1H); 9.45 (broad d, J=5.5 Hz: 1H); 11.50 (broad s: 1H); 11.64 (broad s: 1H).

### EXAMPLE 3

### Preparation of bis[(1-methylquinolinio-6-yl)amido]-2,6-pyrazinedicarboxylic acid diiodide

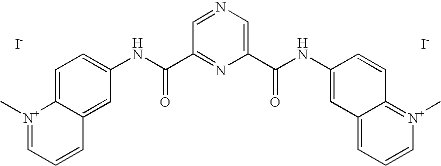

Step 1: The procedure is carried out as in Step 1 of Example 1, but starting with 450 mg of 2,6-pyrazinedicarboxylic acid, which may be prepared according to J. Med. Chem. (1996), 29, 1452-57, 450 mg of 6-aminoquinoline, 600 mg of EDCI and 40 mg of HOBT in 15 ml of dichloromethane and 10 ml of DMF, with stirring overnight at room temperature. After purification by preparative LC/MS, 170 mg of bis-[(quinolin-6-yl)amido]-2,6-pyrazinedicarboxylic acid are obtained in the form of a white powder which is used as it is in the next step.

Step 2: The procedure is carried out as in Step 2 of Example 1, but starting with 50 mg of bis-[(quinolin-6-yl)amido]-2,6-pyrazinedicarboxylic acid, obtained above in 1 ml of DMF and 5 ml of iodomethane. 51 mg of bis[(1-methylquinolinio-6-yl)amido]-2,6-pyrazinedicarboxylic acid diiodide are then obtained, after recrystallization from ethanol, in the form of a pale yellow solid whose characteristics are the following:

Melting point (Kofler)>260° C.

¹H NMR spectrum (300 MHz, (CD₃)₂SO d6, δ in ppm): 4.70 (s: 6H); 8.22 (dd, J=8.5 and 6 Hz: 2H); 8.70 (s: 4H); 9.21 (broad s: 2H); 9.39 (d, J=8.5 Hz: 2H); 9.46 (d, J=6 Hz: 2H); 9.71 (s: 2H); 11.62 (broad s: 2H).

### EXAMPLE 4

### Preparation of bis[(1-methylquinolinio-6-yl)amido]-1,3-benzenedicarboxylic acid diiodide

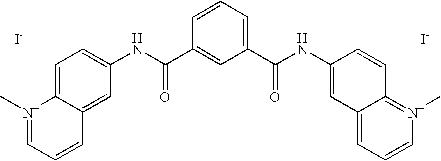

Step 1: The procedure is carried out as in Step 1 of Example 1, but starting with 300 mg of isophthalic acid, 521 mg of 6-aminoquinoline, 727 mg of EDCI and 50 mg of HOBT in 10 ml of DMF, with stirring overnight at room temperature. 593 mg of bis[(quinolin-6-yl)amido]-1,3-benzenedicarboxylic acid are obtained in the form of a white powder which is used as it is in the next step.

Step 2: The procedure is carried out as in Step 2 of Example 1, but starting with 100 mg of bis[(quinolin-6-yl)amido]-1,3-benzenedicarboxylic acid, obtained above in 1 ml of DMF and 6 ml of iodomethane. 72 mg of bis[(1-methylquinolinio-6-yl)amido]-1,3-benzenedicarboxylic acid diiodide are thus obtained, after recrystallization from ethanol, in the form of a pale yellow solid whose characteristics are the following:

Melting point (Kofler)>260° C.

¹H NMR spectrum (300 MHz, (CD₃)₂SO d6, δ in ppm): 4.63 (s: 6H); 7.77 (t, J=7.5 Hz: 1H); 8.07 (dd, J=8.5 and 6 Hz: 2H); 8.29 (broad d, J=7.5 Hz:2H); 8.49 (mt: 4H); 8.84 (broad s: 1H); 8.99 (broad s: 2H); 9.16 (d, J=8.5 Hz: 2H); 9.28 (d, J=6 Hz: 2H).

### EXAMPLE 5

### Preparation of bis[(1-methylquinaldinio-6-yl)amido]-2,6-pyridinedicarboxylic acid diiodide

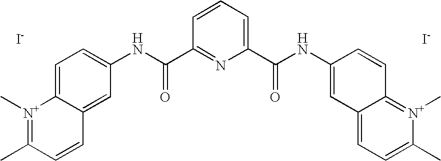

Step 1: The procedure is carried out as in Step 1 of Example 1, but starting with 500 mg of 2,6-pyridinedicarboxylic acid, 945 mg of 6-aminoquinaldine, which may be prepared according to EP 286277, 1.2 g of EDCI and 100 mg of HOBT in 15 ml of dichloromethane and 3 ml of DMF, with stirring for 24 hours at room temperature. 1.47 g of bis[(quinaldin-6-yl)amido]-2,6-pyridinedicarboxylic acid are obtained in the form of a white powder which is used as it is in the next step.

Step 2: The procedure is carried out as in Step 2 of Example 1, but starting with 150 mg of bis[(quinaldin-6-yl)amido]-2,6-pyridinedicarboxylic acid, obtained above in 1.5 ml of DMF and 6 ml of iodomethane. 161 mg of bis[(1-methylquinaldinio-6-yl)amido]-2,6-pyridinedicarboxylic acid diiodide are thus obtained, after recrystallization from ethanol, in the form of a yellow solid whose characteristics are the following:

Melting point (Kofler)>260° C.

### EXAMPLE 6

### Preparation of 2-[(1-methylquinolinio-6-yl)amido]-6-[(4-aminoquinaldinio-6-yl)amido]-2,6-pyridinedicarboxylic acid iodide, isolated in its tautomeric imino form below

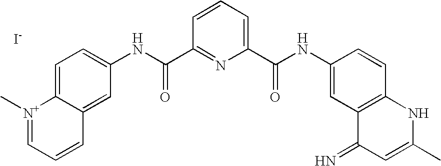

Step 1: The procedure is carried out as in Step 1 of Example 2, but starting with 500 mg of n-butyl monoester of 2,6-pyridinedicarboxylic acid, 388 mg of 4,6-diaminoquinaldine, which may be obtained according to WO 01/40218, 472 mg of EDCI and 30 mg of HOBT in 10 ml of dichloromethane and 5 ml of DMF for 20 hours at room temperature. 450 mg of n-butyl ester of 6-[(4-aminoquinaldin-6-yl)amido]-2,6-pyridinedicarboxylic acid, which is used as it is in the next step, are obtained, after purification by flash chromatography on alumina, eluting with a mixture of dichloromethane and methanol (95/5 by volume).

Step 2: The procedure is carried out as in Step 2 of Example 2, but starting with 450 mg of n-butyl ester of 6-[(4-aminoquinaldin-6-yl)amido]-2,6-pyridinedicarboxylic acid, obtained above, in 20 ml and 1.19 ml of a 1 M aqueous potassium hydroxide solution. 243 mg of 6-[(4-aminoquinaldin-6-yl)amido]-2,6-pyridinedicarboxylic acid are thus obtained in the form of a beige solid which is used as it is in the next step.

Step 3: The procedure is carried out as in Step 3 of Example 2, but starting with 93 mg of 6-[(4-aminoquinaldin-6-yl)amido]-2,6-pyridinedicarboxylic acid, obtained above, 41.6 mg of 6-aminoquinoline, 61 mg of EDCI and 14 mg of HOBT in 5 ml of dichloromethane and 5 ml of DMF for 48 hours at room temperature. 101 mg of 2-[(quinolin-6-yl)amido]-6-[(4-aminoquinaldin-6-yl)amido]-2,6-pyridinedicarboxylic acid are thus obtained in the form of a beige powder which is used as it is in the next step.

Step 4: The procedure is carried out as in Step 2 of Example 1, but starting with 65 mg of 2-[(quinolin-6-yl)amido]-6-[(4-aminoquinaldin-6-yl)amido]-2,6-pyridinedicarboxylic acid, obtained above, in 1 ml of DMF and 5 ml of iodomethane. 58 mg of 2-[(1-methylquinolinio-6-yl)amido]-6-[(4-aminoquinaldinio-6-yl)amido]-2,6-pyridinedicarboxylic acid iodide, isolated in its tautomeric imino form, are thus obtained, after recrystallization from ethanol, in the form of a yellow solid whose characteristics are the following:

Melting point (Kofler)>260° C.

¹H NMR spectrum (400 MHz, (CD₃)₂SO d6, at a temperature of 373K, δ in ppm): 2.64 (s: 3H); 4.70 (s: 3H); 6.71 (s: 1H); 7.95 (d, J=9 Hz: 1H); 8.16 (broad dd, J=8.5 and 5.5 Hz: 1H); from 8.20 to 8.40 (broad unresolved complex: 1H); 8.29 (d, J=8.5 Hz:1H); 8.43 (t, J=7.5 Hz: 1H); 8.53 (mt: 2H); 8.63 (d, J=9.5 Hz: 1H); 8.76 (broad d, J=9.5 Hz: 1H); 8.86 (broad s: 1H); 9.11 (broad s: 1H); 9.30 (d, J=8.5 Hz: 1H); 9.41 (d, J=5.5 Hz: 1H); 11.18 (broad s: 1H); 11.44 (broad s: 1H); from 13.00 to 13.50 (broad unresolved complex: 1H).

### EXAMPLE 7

### Preparation of bis[(1-methylquinaldinio-6-yl)amido]-2,6-benzenedicarboxylic acid diiodide

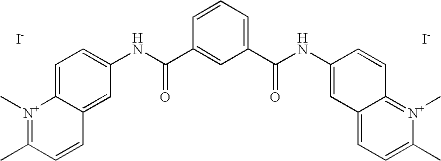

Step 1: The procedure is carried out as in Step 1 of Example 1 but starting with 100 mg of isophthalic acid, 190.5 mg of 6-aminoquinaldine, 242 mg of EDCI and 20 mg of HOBT in 5 ml of DMF, with stirring for 20 hours at room temperature. 265 mg of bis[(quinaldin-6-yl)amido]-2,6-benzenedicarboxylic acid are obtained in the form of a beige powder which is used as it is in the next step.

Step 2: The procedure is carried out as in Step 2 of Example 1, but starting with 95 mg of bis[(quinaldin-6-yl)amido]-2,6-benzenedicarboxylic acid, obtained above, in 1 ml of DMF and 5 ml of iodomethane. 83 mg of bis[(1-methylquinaldinio-6-yl)amido]-2,6-benzenedicarboxylic acid diiodide are thus obtained, after recrystallization from ethanol, in the form of a yellow solid whose characteristics are the following:

Melting point (Kofler)>260° C.

¹H NMR spectrum (300 MHz, (CD₃)₂SO d6, δ in ppm): 3.08 (s: 6H); 4.47 (s: 6H); 7.86 (t, J=8 Hz: 1H); 8.10 (d, J=8.5 Hz: 2H); 8.33 and 8.34 (2d, J=8 Hz:2H in total); 8.48 (dd, J=9.5 and 2.5 Hz: 2H); 8.66 (d, J=9.5 Hz: 2H); 8.70 (broad s: 1H); 8.96 (d, J=2.5 Hz: 2H); 9.13 (d, J=8.5 Hz: 2H); 11.13 (broad s: 2H).

### EXAMPLE 8

### Preparation of bis[(1-methylquinolin-6-yl)amido]-2,4-pyridinedicarboxylic acid diiodide

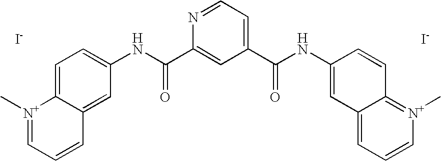

Step 1: The procedure is carried out as in Step 1 of Example 1, but starting with 100 mg of 2,4-pyridinedicarboxylic acid, 181 mg of 6-aminoquinaldine, 241 mg of EDCI and 16 mg of HOBT in 5 ml of DMF, with stirring for 20 hours at room temperature. 325 mg of bis[(quinolin-6-yl)amido]-2,4-pyridinedicarboxylic acid are obtained in the form of a beige powder which is used as it is in the next step.

Step 2: The procedure is carried out as in Step 2 of Example 1, but starting with 300 mg of bis[(quinolin-6-yl)amido]-2,4-pyridinedicarboxylic acid, obtained above, in 3 ml of DMF and 10 ml of iodomethane. 372 mg of bis[(1-methylquinolin-6-yl)amido]-2,4-pyridinedicarboxylic acid diiodide are thus obtained, after recrystallization from ethanol, in the form of a yellow solid whose characteristics are the following:

Melting point (Kofler)>260° C.

¹H NMR spectrum (400 MHz, (CD₃)₂SO d6, at a temperature of 373 K, δ in ppm): 4.61 (s: 3H); 4.68 (s: 3H); 7.93 (dd, J=8.5 and 5.5 Hz: 1H); 8.11 (dd, J=8.5 and 5.5 Hz: 1H); 8.33 (mt: 2H); 8.43 (broad d, J=9.5 Hz: 1H); 8.56 (broad d, J=9.5 Hz: 1H); 8.75 (broad dd, J=9.5 and 1.5 Hz: 1H); 8.79 (d, J=1.5 Hz: 1H); 8.89 (s: 1H); 8.90 (mt: 1H); 8.99 (broad d, J=8.5 Hz: 1H); 9.10 (mt: 1H); 9.10 (s: 1H); 9.23 (d, J=8.5 Hz: 1H); 9.35 (d, J=5.5 Hz: 1H).

### EXAMPLE 9

### Preparation of 2-[(1-methylquinolinio-6-yl)amido]-6-[(1-methylquinolinio-3-yl)amido]-2,6-pyridinedicarboxylic acid iodide,

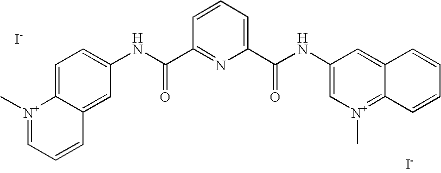

Step 1: 1.74 g of 2,6-pyridinedicarboxylic acid, 500 mg of 4-aminoquinoline, 543 μL of N-(2-chloroethyl)diisopropylamine hydrochloride (DIC) and 469 mg of HOBT are dissolved, in a 25 ml three-necked round-bottomed flask, in 15 ml of DMF. As soon as disappearance is observed in TLC (60F₂₅₄, silica plate, eluent dichloromethane/methanol 90/10), the reaction medium is deposited on a cartridge of 5 g of sulfonic resin (40 μM Varian Mega Bond Elut SCX model). The fraction, eluted with a 0.1 M solution of ammoniacal methanol, is concentrated under reduced pressure. The residue is taken up in 5 ml of dichloromethane, and the precipitate formed is drained and then washed with a 1 M aqueous hydrochloric acid solution. 510 mg of 2-[(quinolin-6-yl)amido]-2,6-pyridinedicarboxylic acid are thus obtained, which acid is used as it is in the next step.

Step 2: The procedure is carried out as in Step 3 of Example 2, but starting with 150 mg of 2-[(quinolin-6-yl)amido]-2,6-pyridinedicarboxylic acid, obtained above, 74 mg of 3-aminoquinoline, which may be prepared according to Tetrahedron. Lett. 2001, 42, 3251-54, 109 mg of EDCI and 7 mg of HOBT in 5 ml of dichloromethane and 5 ml of DMF for 48 hours at room temperature. 180 mg of 2-[(quinolin-6-yl)amido]-6-[(quinolin-3-yl)amido]-2,6-pyridinedicarboxylic acid are thus obtained in the form of a pink beige powder which is used as it is in the next step.

Step 3: The procedure is carried out as in Step 2 of Example 1, but starting with 150 mg of 2-[(quinolin-6-yl)amido]-6-[(quinolin-3-yl)amido]-2,6-pyridinedicarboxylic acid, obtained above, in 1 ml of DMF and 5 ml of iodomethane. 162 mg of 2-[(1-methylquinolinio-6-yl)amido]-6-[(1-methylquinolinio-3-yl)amido]-2,6-pyridinedicarboxylic acid iodide are thus obtained, after recrystallization from ethanol, in the form of a yellow solid whose characteristics are the following:

Melting point (Kofler)>260° C.

¹H NMR spectrum (300 MHz, (CD₃)₂SO d6, δ in ppm): 4.68 (broad s: 3H); 4.78 (broad s: 3H); 8.07 (broad t, J=7.5 Hz: 1H); from 8.15 to 8.30 (mt: 1H); 8.20 (dd, J=8.5 and 5.5 Hz: 1H); from 8.40 to 8.65 (mt: 5H); 8.65 (broad d, J=9.5 Hz: 1H); 8.75 (broad dd, J=9.5 and 2 Hz: 1H); 9.22 (broad d, J=2 Hz: 1H); 9.35 (broad d, J=8.5 Hz: 1H); 9.43 (broad d, J=5.5 Hz: 1H); 9.64 (broad s: 1H); 10.10 (unresolved complex: 1H); from 11.10 to 12.50 (broad unresolved complex: 2H).

### EXAMPLE 10

### Preparation of 2-[(1-methylquinolinio-6-yl)amido]-6-[(1-methylquinolinio-5-yl)amido]-2,6-pyridinedicarboxylic acid iodide

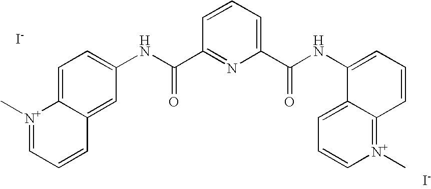

Step 1: The procedure is carried out as in Step 3 of Example 2, but starting with 150 mg of 2-[(quinolin-6-yl)amido]-2,6-pyridinedicarboxylic acid, obtained in Step 1 of Example 9, 74 mg of 5-aminoquinoline, 109 mg of EDCI and 7 mg of HOBT in 5 ml of dichloromethane and 5 ml of DMF for 48 hours at room temperature. 201 mg of 2-[(quinolin-6-yl)amido]-6-[(quinolin-5-yl)amido]-2,6-pyridinedicarboxylic acid are thus obtained in the form of a beige powder which is used as it is in the next step.

Step 2: The procedure is carried out as in Step 2 of Example 1, but starting with 180 mg of 2-[(quinolin-6-yl)amido]-6-[(quinolin-5-yl)amido]-2,6-pyridinedicarboxylic acid, obtained above, in 1.5 ml of DMF and 5 ml of iodomethane. 203 mg of 2-[(1-methylquinolinio-6-yl)amido]-6-[(1-methylquinolinio-5-yl)amido]-2,6-pyridinedicarboxylic acid iodide are thus obtained, after recrystallization from ethanol, in the form of a yellow solid whose characteristics are the following:

Melting point (Kofler)>260° C.

¹H NMR spectrum (400 MHz, (CD₃)₂SO d6, at a temperature of 373 K, δ in ppm): 4.69 (broad s: 6H); 8.08 (very broad dd, J=8.5 and 5 Hz: 1H); 8.14 (dd, J=8.5 and 5 Hz: 1H); 8.23 (unresolved complex: 1H); from 8.30 to 8.45 (mt: 2H); 8.49 (mt: 2H); 8.54 (dd, J=8 and 0.5 Hz: 1H); 8.61 (d, J=9.5 Hz: 1H); 8.70 (dd, J=9.5 and 2.5 Hz: 1H); 9.07 (d, J=2.5 Hz: 1H); 9.26 (d, J=8.5 Hz: 1H); 9.38 (d, J=5 Hz: 1H); 9.45 (broad d, J=5 Hz: 1H); 9.60 (broad d, J=8.5 Hz: 1H).

### EXAMPLE 11

### Preparation of bis[(1-methylquinolinio-3-yl)amido]-2,6-pyridinedicarboxylic acid diiodide

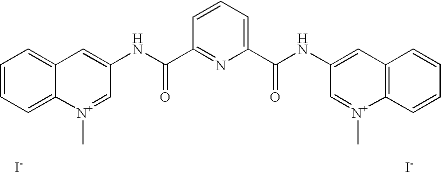

Step 1: The procedure is carried out as in Step 1 of Example 1, but starting with 150 mg of 2,6-pyridinedicarboxylic acid, 945 mg of 3-aminoquinoline, which may be prepared according to Tetrahedron. Lett. 2001, 42, 3251-54, 361 mg of EDCI and 24 mg of HOBT in 10 ml of DMF, with stirring for 18 hours at room temperature. 495 mg of bis[(quinolin-3-yl)amido]-2,6-pyridinedicarboxylic acid are obtained in the form of a white powder which is used as it is in the next step.

Step 2: The procedure is carried out as in Step 2 of Example 1, but starting with 200 mg of bis[(quinolin-3-yl)amido]-2,6-pyridinedicarboxylic acid, obtained above in 3 ml of DMF and 10 ml of iodomethane. 231 mg of bis[(1-methylquinolinio-3-yl)amido]-2,6-pyridinedicarboxylic acid diiodide are thus obtained, after recrystallization from ethanol, in the form of a yellow solid whose characteristics are the following:

Melting point (Kofler)>260° C.

¹H NMR spectrum (300 MHz, (CD₃)₂SO d6, δ in ppm): 4.82 (broad s: 6H); 8.12 (broad t, J=8 Hz: 2H); 8.27 (broad t, J=8 Hz: 2H); from 8.45 to 8.65 (mt: 7H); 9.68 (broad s: 2H); 10.14 (broad s: 2H); 11.93 (unresolved complex: 2H).

### EXAMPLE 12

### Preparation of 2-[(1-methylquinolinio-6-yl)amido]-6-[2(-1-methylpiperidinio-1-yl)ethylamido]-2,6-pyridinedicarboxylic acid diiodide

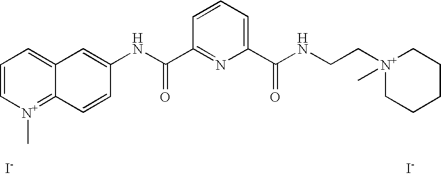

Step 1: The procedure is carried out as in Step 3 of Example 2, but starting with 140 mg of 2-[(quinolin-6-yl)amido]-2,6-pyridinedicarboxylic acid, obtained in Step 1 of Example 9, 73 μl of 1-(2-aminoethyl)piperidine, 100 mg of EDCI and 7 mg of HOBT in 5 ml of dichloromethane and 5 ml of DMF for 48 hours at room temperature. 200 mg of 2-[(quinolin-6-yl)amido]-6-[2-(piperidin-1-yl)ethylamido]-2,6-pyridinedicarboxylic acid are thus obtained, after purification by flash chromatography on silica gel, eluting with a mixture of dichloromethane and methanol (80/20 by volume), in the form of a yellow oil which is used as it is in the next step.

Step 2: The procedure is carried out as in Step 2 of Example 1, but starting with 200 mg of 2-[(quinolin-6-yl)amido]-6-[2-(piperidin-1-yl)ethylamido]-2,6-pyridinedicarboxylic acid, obtained above, in 2 ml of DMF and 6 ml of iodomethane. 73 mg of 2-[(1-methylquinolinio-6-yl)amido]-6-[2(-1-methylpiperidinio-1-yl)ethylamido]-2,6-pyridinedicarboxylic acid iodide are thus obtained, after recrystallization from ethanol, in the form of a yellow solid whose characteristics are the following

Melting point (Kofler)>260° C.

### EXAMPLE 13

### Preparation of 2,6-pyridinedicarboxylic acid 2-[(1-methylquinolinio-3-yl)amide]-6-[quinolin-3-yl)amide]iodide

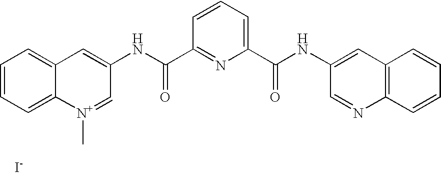

106 mg of 2,6-pyridinedicarboxylic acid bis[(quinolin-3-yl)amide], obtained in step 1 of Example 11, are dissolved in 3 mL of DMF, 36.6 mg of methyl iodide are then added and the mixture is heated in a 10 mL sealed tube for 5 hours at 80° C. After cooling, 10 mL of diethyl ether are added. The precipitate obtained is suction-filtered, washed with diethyl ether and then purified by LC/MS chromatography using a C18 Waters Xterra 3.5 μM silica column 3 mm in diameter and 50 mm long, eluting with a linear elution gradient consisting at time 0 (t0=0 min) of water supplemented with 0.05% TFA and at the finished time (tf=4 min) of acetonitrile containing 0.05% TFA. By collecting the fraction eluted at 2.63 min, 98 mg of 2,6-pyridinedicarboxylic acid 2-[(1-methylquinolinio-3-yl)amide]-6-[quinolin-3-yl)amide]iodide are obtained in the form of a yellow solid, the characteristics of which are as follows:

melting point (Kofler)>260° C.

### EXAMPLE 14

### Preparation of 2,6-pyridinedicarboxylic acid 2-[(1-methylquinolinio-3-yl)amide]-6-[1-(2-hydroxyethyl)quinolinio-3-yl)amide]iodide

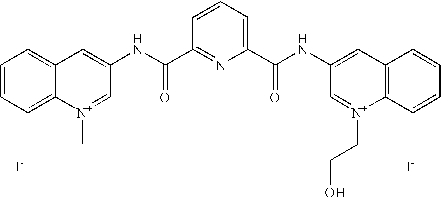

112.3 mg of 2,6-pyridinedicarboxylic acid 2-[(1-methylquinolinio-3-yl)amide]6-[quinolin-3-yl)amide]iodide, obtained as in Example 13, are suspended in 5 mL of acetonitrile in a 25 ml three-necked flask, 1 mL of 2-iodoethanol is added and the mixture is then refluxed for 72 hours. The insoluble red material obtained is suction-filtered, washed with acetonitrile and then with a mixture of acetonitrile and ethanol (50/50 by volume) and then purified by LC/MS chromatography using a C18 Waters Xterra 3.5 μM silica column, 3 mm in diameter and 50 mm long, eluting with a linear elution gradient consisting at time 0 (t0=0 min) of water supplemented with 0.05% TFA and at the finished time (tf=4 min) of acetonitrile containing 0.05% TFA. By collecting the fraction eluted at 2.20 min, 30 mg of 2,6-pyridinedicarboxylic acid 2-[(1-methylquinolinio-3-yl)amide]-6-[1-(2-hydroxyethyl)quinolinio-3-yl)amide]iodide, are obtained in the form of a beige-colored solid, the characteristics of which are as follows:

melting point (Kofler)>260° C.

### EXAMPLE 15

### Preparation of 4-bromo-2,6-pyridinedicarboxylic acid bis[(1-methylquinolinio-3-yl)amide]diiodide

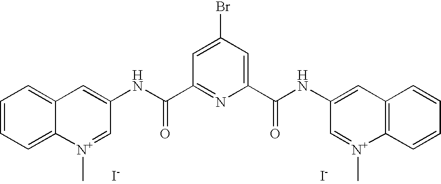

Step 1: The process is performed as in step 1 of Example 1, but starting with 502 mg of 4-bromo-2,6-pyridinedicarboxylic acid, which may be prepared according to Tetrahedron Lett. 2001, 42, 4849-51, and 588 mg of 3-aminoquinoline, which may be prepared according to Tetrahedron Lett. 2001, 42, 3251-54, 821 mg of EDCI and 55 mg of HOBT in 23 mL of DMF, with stirring for 20 hours at room temperature. 935 mg of 4-bromo-2,6-pyridinedicarboxylic acid bis[(quinolin-3-yl)amide] are obtained in the form of a green-grey powder, which is used without further purification in the following step.

Step 2: The process is performed as in step 2 of Example 1, but starting with 300 mg of 4-bromo-2,6-pyridinedicarboxylic acid bis[(quinolin-3-yl)amide], obtained above, in 8 mL of DMF and 12.63 mL of iodomethane. After recrystallization from ethanol, 296 mg of 4-bromo-2,6-pyridinedicarboxylic acid bis[(1-methylquinolinio-3-yl)amide]diiodide are thus obtained in the form of a yellow solid, the characteristics of which are as follows:

melting point (Kofler)>260° C.

### EXAMPLE 16

### Pharmaceutical Composition

Tablets corresponding to the following formula were prepared:

| | | |
|---|---|---|
| Product of Example 1 | 0.2 | g |
| Excipient for a finished tablet containing | 1 | g |
| (details of the excipient: lactose, talc, starch, | | |
| magnesium stearate). | | |

### EXAMPLE 17

### Pharmaceutical Composition

Tablets corresponding to the following formula were prepared:

| | | |
|---|---|---|
| Product of Example 12 | 0.2 | g |
| Excipient for a finished tablet containing | 1 | g |
| (details of the excipient: lactose, talc, starch, | | |
| magnesium stearate). | | |

1. A compound which binds the G-quadruplex structure of DNA or RNA, which corresponds to the following general formula (IB):
nitrogen-containing aromatic ring possessing a nitrogen atom in quaternary form-(NR₃)p-CO-distribution agent-(CO)m-(NR′₃)-X-aromatic or nonaromatic ring
(IB)
wherein m, p and q, which are identical or different, represent the integer 0 or 1,
wherein
the nitrogen-containing aromatic ring possessing a quaternary atom is:
a quinoline optionally substituted with at least
one group N(Ra)(Rb) wherein Ra and Rb, are identical or different, are hydrogen or C1-C4 alkyl or
C1-C4 alkyl or alkoxy or
wherein the nitrogen atom is quaternized with a C1-C4 alkyl chain optionally substituted with hydroxyl, carboxyl, C1-C4 alkoxy, C1-C4 alkylthio, amino, C1-C4 alkylamino or C1-C4 dialkylamino for each alkyl group;
the aromatic or nonaromatic ring is:
a quinoline optionally substituted with at least
one group N(Ra)(Rb) wherein Ra and Rb, are identical or different, are hydrogen or C1-C4 alkyl or
C1-C4 alkyl or alkoxy, or
a quinoline possessing a nitrogen in quaternary form, or
a benzamidine, or
a pyridine, or
a phenyl nucleus optionally substituted with halogen, C1-C4 alkoxy, cyano, or carbonylamino wherein said carbonylamino group is optionally substituted with one or more substituents independently selected from the group consisting of C1-C4 alkyl, guanyl, C1-C4 alkylthio, amino, C1-C4 alkylamino, a C1-C4 dialkylamino for each alkyl group, nitro, C1-C4 alkyleneamino and C2-C4 alkenyleneamino, or
a mono- or bi- or tricyclic aromatic or nonaromatic heterocyclic nucleus containing 0 to 2 heteroatoms per ring provided that at least one heteroatom has at least one ring optionally substituted with one or more substituents selected from the group consisting of C1-C4 alkyl, C1-C4 alkylene and C2-C4 alkenylene, and when the heteroatom, is nitrogen, it may be optionally in quaternary form;
R₃ and R′₃, which are identical or different, are independently hydrogen, C1-C4 alkyl or an aralkyl radical wherein the alkyl part is C1-C4;
X represents a single bond, C1-C4 alkyl, C2-C4 alkenyl, a C2-C4 alkynyl or phenyl;
the distribution agent is:
a 5- or 6-membered heterocyclic radical containing a sulfur, oxygen or nitrogen,
a phenyl, or
a diazine or triazine group,
wherein heterocyclic, phenyl, diazine or triazine groups are optionally substituted with one or more substituents chosen from halogen, C1-C4 alkyl, thio, oxy or amino substituents wherein any such substituents are optionally substituted with one or more alkyl chains containing 1 to 4 carbon atoms,
it being understood that for the compounds of formula (IB) in which X represents a single bond, when the distribution agent represents phenyl optionally substituted with NH₂, m, p and q represent 1 and R₃ and R′₃ represent hydrogen, then the nitrogen-containing aromatic ring and the aromatic ring do not both represent a quinoline which is unsubstituted or substituted on its nitrogen atom with an alkyl radical containing 1 to 6 carbon atoms,
or one of its salts and when the distribution agent represents a triazine and p and q both represent the integer 1 then m does not represent the integer 0;
said compounds of formula (IB) may be in all the possible isomeric forms; or an addition salt with an inorganic or organic acid or with an inorganic or organic base of said compound of formula (IB); or a prodruq of said compound of formula (IB).

2. The compound according to claim 1 which binds to the G-quadruplex structure of telomers.

3. The compound according to claim 1 which is a highly specific ligand for G-quadruplex DNA.

4. The compound according to claim 1, wherein the heterocyclic groups among which the distribution agent may be chosen are thienyl and pyridyl groups.

5. The compound according to claim 1, wherein the distribution agent is chosen from the heterocyclic groups pyridyl, thienyl, a phenyl, a diazine or a triazine.

6. The compound according to claim 5, wherein the diazine groups are pyrazines.

7. The compound according to claim 1, wherein the distribution agent is meta-disubstituted with the groups “nitrogen-containing aromatic ring possessing a nitrogen atom in quaternary form-(NR₃)p-CO” and “(CO)m-(NR′₃)q-aromatic or nonaromatic ring” as defined in claim 1 and wherein the distribution agent is optionally substituted by halogen.

8. The compound according to claim 1, wherein the heterocycle in quaternary form is a quinoline.

9. The compound according to claim 1, wherein the distribution agent represents a pyridine which is 2,6-disubstituted or a pyridazine which is 2-6-disubstituted with the groups “nitrogen-containing aromatic ring possessing a quaternary nitrogen atom in quaternary form-(NR₃)p-CO” and “(CO)m-(NR′₃)q-aromatic or nonaromatic ring” and wherein the quaternized heterocycle is an N-methylquinolinium and wherein the distribution agent is optionally substituted by halogen.

10. The compound according to claim 1, wherein p and q are the integer 1.

11. The compound according to claim 1, wherein m, p and q are the integer 1.

12. The compound according to claim 1, having the formula (Ia) 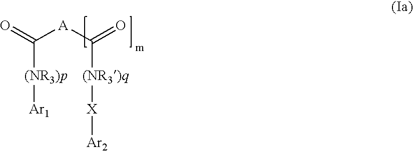
wherein m, p and q, are identical or different integers from 0 to 1
wherein
A is:
a 5- or 6-membered heterocyclic radical containing a sulfur, oxygen or nitrogen,
a phenyl, or
a diazine or triazine group,
wherein the heterocyclic, phenyl, diazine or triazine radicals are optionally substituted with one or more substituents chosen from halogen, C1-C4 alkyl, thio, oxy or amino substituents wherein any such substituents are optionally substituted with one or more short-chain alkyl chains containing 1 to 4 carbon atoms;
Ar₁ and Ar₂, may be the same or different
wherein Ar₁ and Ar₂ are identical, they are a nitrogen-containing aromatic ring possessing a quaternary atom represented by a quinoline optionally substituted with at least
one group N(Ra)(Rb) wherein Ra and Rb, are identical or different, are hydrogen or C1-C4 alkyl or
one C1-C4 alkyl or alkoxy group, or
wherein the nitrogen atom is quaternized with a C1-C4 alkyl chain optionally substituted with a hydroxyl, carboxyl, C1-C4 alkoxy, C1-C4 alkylthio, amino, C1-C4 alkylamino or C1-C4 dialkylamino for each alkyl group;
wherein Ar₁ and Ar₂ are different
Ar₁ represents one of the above possibilities for Ar₁ and Ar₂ represents
phenyl optionally substituted with halogen, C1-C4 alkoxy, cyano, carbonylamino wherein said carbonylamino is optionally substituted with one or more substituents independently selected from the group consisting of C1-C4 alkyl, a guanyl, a C1-C4 alkylthio, amino, C1-C4 alkylamino, C1-C4 dialkylamino for each alkyl group, nitro, C1-C4 alkyleneamino and C2-C4 alkenyleneamino, or
a benzamidine,
a pyridyl,
a mono- or bi- or tricyclic aromatic or nonaromatic heterocyclic nucleus containing 0 to 2 heteroatoms per ring provided that at least one heteroatom has at least one ring optionally substituted with one or more substituents independently selected from C1-C4 alkyl C1-C4 alkylene and C2-C4 alkenylene;
R₃ and R′₃, are identical or different, are independently hydrogen, C1-C4 alkyl or aralkyl wherein alkyl is C1-C4;
X is a single bond, or C1-C4 alkyl, a C2-C4 alkenyl, alkynyl or phenyl; said compound of formula (Ia) may be in all the possible isomeric forms; or an addition salt with an inorganic or organic acid or with an inorganic or organic base of said compound of formula (Ia) or a prodrug of said compound of formula (Ia).

13. The compound of formula (Ia) according to claim 12 wherein X is C1-C4 alkyl, the other substituents of the compound of formula (Ia) being as defined in claim 12, said compound of formula (Ia) may be in all the possible isomeric forms; or an addition salt with an inorganic or organic acid or with an inorganic or organic base of said compound of formula (Ia).

14. The compound according to claim 12, wherein A is chosen from the heterocyclic groups pyridyl or thienyl, a phenyl, a diazine or a triazine.

15. The compound according to claim 14, wherein the diazine groups which A can represent are pyrazines.

16. The compound according to claim 12, wherein A is meta-disubstituted with the groups “nitrogen-containing aromatic ring possessing a nitrogen atom in quaternary form-(NR₃)p-CO” and “(CO)m-(NR′₃)q-aromatic or nonaromatic ring”, and wherein A is optionally substituted by halogen.

17. The compound according to claim 12, wherein the heterocycle in quaternary form is a quinoline.

18. The compound according to claim 12, wherein A represents a pyridine which is 2,6-disubstituted or a pyridazine which is 2-6-disubstituted with the groups “nitrogen-containing aromatic ring possessing a quaternary nitrogen atom in quaternary form-(NR₃)p-CO” and “(CO)m-(NR′₃)q-aromatic or nonaromatic ring” and wherein the quaternized heterocycle is an N-methylquinolinium, and wherein A is optionally substituted by halogen.

19. The compound according to claim 12, wherein m, p and q are the integer 1.

20. The compound according to claim 12, wherein p and q are the integer 1.

21. The compound according to claim 12, wherein Ar₂ is independently selected from the group consisting of 4-amino- or 4-methylamino-, 4-dimethylamino- or 4-alkoxy-quinolyl and -quinolinium, wherein said quinolinium is optionally substituted with one or two methyl groups.

22. The compound according to claim 12, wherein R₃ and R′₃ are hydrogen.

23. The compound according to claim 1, selected from the group consisting of:
bis[(1-methylquinolinio-6-yl)amido]-2,6-pyridinedicarboxylic acid diiodide;
2-[(1-methylquinolinio-6-yl)amido]-6-[(4-dimethylamino-1-methylquinaldinio-6-yl)amido]-2,6-pyridinedicarboxylic acid diiodide;
bis[(1-methylquinolinio-6-yl)amido]-2,6-pyrazinedicarboxylic acid diiodide;
bis[(1-methylquinolinio-6-yl)amido]-1,3-benzenedicarboxylic acid diiodide;
bis[(1-methylquinaldinio-6-yl)amido]-2,6-pyridinedicarboxylic acid diiodide;
2-[(1-methylquinolinio-6-yl)amido]-6-[(4-aminoquinaldinio-6-yl)amido]-2,6-pyridinedicarboxylic acid iodide;
bis[(1-methylquinaldinio-6-yl)amido]-2,6-benzenedicarboxylic acid diiodide;
bis[(1-methylquinolin-6-yl)amido]-2,4-pyridinedicarboxylic acid diiodide;
2-[(1-methylquinolinio-6-yl)amido]-6-[(1-methylquinolinio-3-yl)amido]-2,6-pyridinedicarboxylic acid iodide;
2-[(1-methylquinolinio-6-yl)amido]-6-[(1-methylquinolinio-5-yl)amido]-2,6-pyridinedicarboxylic acid iodide;
bis[(1-methylquinolinio-3-yl)amido]-2,6-pyridinedicarboxylic acid diiodide;
2-[(1-methylquinolinio-6-yl)amido]-6-[2(-1-methylpiperidinio-1-yl)ethylamido]-2,6-pyridinedicarboxylic acid diiodide;
2,6-pyridinedicarboxylic acid 2-[(1-methylquinolinio-3-yl)amide]-6-[quinolin-3-yl)amide]iodide;
2,6-pyridinedicarboxylic acid 2-[(1-methylquinolinio-3-yl)amide]-6-[1-(2-hydroxyethyl)quinolinio-3-yl)amide]iodide; and
4-bromo-2,6-pyridinedicarboxylic acid bis[(1-methylquinolinio-3-yl)amide]diiodide,
said compound may be in all the possible isomeric forms; or an addition salt with an inorganic or organic acid or with an inorganic or organic base of said compound.

24. The compound according to claim 1, which is selected from the group consisting of:
bis[(1-methylquinolinio-6-yl)amido]-2,6-pyridinedicarboxylic acid diiodide;
2-[(1-methylquinolinio-6-yl)amido]-6-[(4-dimethylamino-1-methylquinaldinio-6-yl)amido]-2,6-pyridinedicarboxylic acid diiodide;
bis[(1-methylquinolinio-6-yl)amido]-2,6-pyrazinedicarboxylic acid diiodide,
bis[(1-methylquinolinio-6-yl)amido]-1,3-benzenedicarboxylic acid diiodide;
bis[(1-methylquinaldinio-6-yl)amido]-2,6-pyridinedicarboxylic acid diiodide;
2-[(1-methylquinolinio-6-yl)amido]-6-[(4-aminoquinaldinio-6-yl)amido]-2,6-pyridinedicarboxylic acid iodide;
bis[(1-methylquinaldinio-6-yl)amido]-2,6-benzenedicarboxylic acid diiodide;
bis[(1-methylquinolin-6-yl)amido]-2,4-pyridinedicarboxylic acid diiodide;
2-[(1-methylquinolinio-6-yl)amido]-6-[(1-methylquinolinio-3-yl)amido]-2,6-pyridinedicarboxylic acid iodide;
2-[(1-methylquinolinio-6-yl)amido]-6-[(1-methylquinolinio-5-yl)amido]-2,6-pyridinedicarboxylic acid iodide;
bis[(1-methylquinolinio-3-yl)amido]-2,6-pyridinedicarboxylic acid diiodide;
2,6-pyridinedicarboxylic acid 2-[(1-methylquinolinio-3-yl)amide]-6-[quinolin-3-yl)amide]iodide;
2,6-pyridinedicarboxylic acid 2-[(1-methylquinolinio-3-yl)amide]-6-[1-(2-hydroxyethyl)quinolinio-3-yl)amide]iodide; and
4-bromo-2,6-pyridinedicarboxylic acid bis[(1-methylquinolinio-3-yl)amide]diiodide,
said compound may be in all the possible isomeric forms; or an addition salt with an inorganic or organic acid or with an inorganic or organic base of said compound.

25. The compound according to claim 1 which is 2-[(1-Methylquinolinio-6-yl)amido]-6-[2(-1-methylpiperidinio-1-yl)ethylamido]-2,6-pyridinedicarboxylic acid diiodide wherein said compound may be in all the possible isomeric forms; or an addition salt with an inorganic or organic acid or with an inorganic or organic base.

26. The compound according to claim 1, which has a telomerase inhibiting activity.

27. The compound according to claim 1, which has an anticancer activity.

28. The compound according to claim 1 having the formula (IB) or a prodrug thereof, said compound of formula (IB) may be in all the possible isomeric forms; or an addition salt with pharmaceutically acceptable inorganic or organic acid or with an inorganic or organic base of formula (IB).

29. The compound according to claim 12 having the formula (Ia) or a prodrug thereof, said compound of formula (Ia) may be in all the possible isomeric forms; or an addition salt with a pharmaceutically acceptable inorganic or organic acid or with an inorganic or organic base of said compound of formula (Ia).

30. A pharmaceutical composition comprising an effective cancer inhibiting amount of a compound of claim 1.

31. The pharmaceutical composition according to claim 30, further comprising active ingredients of other chemotherapy medicaments against cancer.

32. The method according to claim 37 wherein the compound according to claim 1 is administered in combination with another anticancer compound.

33. The method as claimed in claim 32, wherein the anticancer compound is selected from the group consisting of alkylating agents, platinum derivatives, antibiotics, antimicrotubule agents, anthracyclines, groups I and II topoisomerases, fluoropyrimidines, cytidine analogs, adenosine analogs, L-asparaginase, hydroxyurea, trans-retinoic acid, suramin, irinotecan, topotecan, dexrazoxane, amifostin, herceptin, estrogen hormones androgen hormones, and antivascular agents.

34. The method according to claim 33, wherein each of the compounds or treatments is administered simultaneously, separately or sequentially.

35. The method according to claim 37 wherein the compound according to claim 1 is administered in combination with radiation.

36. The method according to claim 35, wherein each of the compounds or treatments is administered simultaneously, separately or sequentially

37. A method of treating a disease selected from the group consisting of cancers, genetic diseases and pilosity diseases which comprises administering to a patient in need of said treatment an effective amount of a compound according to claim 1 having the formula (IB) or a pharmaceutically acceptable salt thereof.

38. The method according to claim 37 wherein the disease is cancer.

39. The method according to claim 38 wherein said cancer is selected from the group consisting of cancer of the breast, stomach, colon, lungs, ovaries, uterus, brain, kidney, larynx, lymphatic system, thyroid, urogenital tract, bones, pancreas, and melanomas.

40. The method according to claim 39 wherein said cancer is cancer of the breast, colon or lungs.

41. The method according to claim 37 wherein said genetic diseases are selected from the group consisting of Bloom's syndrome, Werner's syndrome, Rothmund-Thomson syndrome and ataxia telangiectasia syndrome.

42. The method of claim 37 wherein said pilosity disease is hyperpilosity.


SEQUENCE LISTING (23 sequences)

SEQ ID NO: 1 - DNA, 37 bp, Artificial
  synthetic oligonucleotide
ggtttaaaataattgagggttagggttagggttaggg

SEQ ID NO: 2 - DNA, 37 bp, Artificial
  synthetic oligonucleotide
ggtttaaaaaatttgccctaaccctaaccctaaccct

SEQ ID NO: 3 - DNA, 37 bp, Artificial
  synthetic oligonucleotide
ggtttaaaataattgaggcttaccgttaccgttacgg

SEQ ID NO: 4 - DNA, 37 bp, Artificial
  synthetic oligonucleotide
ggtttaaaaaatttgcggtaacggtaacggtaagcct

SEQ ID NO: 5 - DNA, 21 bp, Artificial
  synthetic oligonucleotide
gggttagggttagggttaggg

SEQ ID NO: 6 - DNA, 13 bp, Artificial
  synthetic oligonucleotide
gaaagagaggagg

SEQ ID NO: 7 - DNA, 30 bp, Artificial
  synthetic oligonucleotide
cctcctctctttcccttctttctctcctcc

SEQ ID NO: 8 - DNA, 13 bp, Artificial
  synthetic oligonucleotide
cctcctctctttc

SEQ ID NO: 9 - DNA, 30 bp, Artificial
  synthetic oligonucleotide
gaaagagaggaggccttggaggagagaaag

SEQ ID NO: 10 - DNA, 30 bp, Artificial
  synthetic oligonucleotide
gaaagagaggaggccttggtggtgtgtttg

SEQ ID NO: 11 - DNA, 24 bp, Artificial
  synthetic oligonucleotide
gagagagagagagagagagagaga

SEQ ID NO: 12 - DNA, 24 bp, Artificial
  synthetic oligonucleotide
aaaaaaaaaataattttaaatatt

SEQ ID NO: 13 - DNA, 24 bp, Artificial
  oligonucleotide probe
ttttttttttattaaaatttataa

SEQ ID NO: 14 - DNA, 24 bp, Artificial
  synthetic oligonucleotide
ctgctgctgctgctgctgctgctg

SEQ ID NO: 15 - DNA, 26 bp, Artificial
  synthetic oligonucleotide
caatcggatcgaattcgatccgattg

SEQ ID NO: 16 - DNA, 22 bp, Artificial
  synthetic oligonucleotide
ccctaaccctaaccctaaccct

SEQ ID NO: 17 - DNA, 22 bp, Artificial
  oligonucleotide probe
agggttagggttagggttaggg

SEQ ID NO: 18 - DNA, 24 bp, Artificial
  synthetic oligonucleotide
ttggggggggggggggggggggtt

SEQ ID NO: 19 - DNA, 23 bp, Artificial
  synthetic oligonucleotide
gggattgggattgggattgggtt

SEQ ID NO: 20 - DNA, 27 bp, Artificial
  synthetic oligonucleotide
gtgcccttacccttacccttaccctaa

SEQ ID NO: 21 - DNA, 36 bp, Artificial
  synthetic oligonucleotide
attccgtcgagcagagttaaaaggccgagaagcgat

SEQ ID NO: 22 - DNA, 18 bp, Artificial
  synthetic oligonucleotide
aatcgttcgagcagagtt

SEQ ID NO: 23 - DNA, 18 bp, Artificial
  synthetic oligonucleotide
atcgcttctcggcctttt